(19) United States
(12) Patent Application Publication
(10) Pub. No.: US 2004/0117874 A1
(43) Pub. Date: Jun. 17, 2004

(54) Methods for accumulating translocated proteins

(76) Inventor: Jianjun Yang, Hockessin, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/685,105

(22) Filed: Oct. 14, 2003

Related U.S. Application Data
(60) Provisional application No. 60/418,585, filed on Oct. 15, 2002.

(51) Int. Cl. A01H 1/00; C12N 15/82; C12N 5/04

(52) U.S. Cl. 800/287; 435/69.1; 435/468; 435/419

(57) ABSTRACT

The present invention provides methods for the accumulation of translocated proteins through targeting to the apoplast, ER lumen, and vacuoles of plants. The methods proceed by providing a consisting of a DNA construct comprising a sporamin signal sequence and a target protein, and optionally, a sporamin propeptide and/or an ER retention signal, according to the specific tissue of the plant in which the translocated protein is expressed and targeted. The work disclosed herein represents the first systematic determination of each respective location's capacity for translocated protein accumulation in different plant tissues.

### FIELD OF INVENTION

The present invention relates to the field of molecular biology and plant genetics. More specifically, this invention relates to methods for the accumulation of high levels of transgene encoded protein in leaf and seed tissues of plants through targeted protein translocation.

### BACKGROUND OF THE INVENTION

The expression of foreign proteins in plants is currently possible in a wide range of dicotyledonous and monocotyledonous species. Foreign protein expression has primarily focused on the improvement of agronomic traits, such as herbicide tolerance, disease and pest resistance, and altered crop quality. In these cases, the improved trait is produced by the expression or suppression of a specific protein. In addition to improving agronomic traits, however, it is also desirable to engineer plants for the purpose of producing proteins to obtain a protein product. The use of plants as protein production systems offers several advantages over non-plant protein production systems. For example, plants in many cases are inexpensive to produce. In addition, plants are capable of storing proteins stably in a variety of specialized organs (e.g., seeds, tubers). Despite these and other attractive features, plants have not been widely used as hosts for production of proteins on a commercial scale.

One reason that plants have not been widely used as hosts for the production of protein products is that, by conventional approaches, transgenic plants typically express and accumulate recombinant proteins at levels below 1% of the total soluble protein. It is desirable to increase the level of accumulated protein to reduce production costs and to reduce costs of downstream processing steps such as transportation and purification.

Thus far, two methods have been traditionally undertaken to increase the expression level of recombinant proteins in transgenic plants. The first is enhancement of the transgene's transcription activity. Unfortunately, significant protein accumulation necessary for commercialization as a product is difficult to achieve using this technique. Attempts have also been made to increase expression levels by targeting the foreign proteins into specific cellular compartments of the plant. By targeting the protein to specific locations, undesirable degradation of the protein can be reduced. This enables greater translocated protein accumulation.

Two major protein secretion pathways, the endoplasmic reticulum (ER) lumen retention pathway and the Golgi body translocation pathway, have been used to direct storage proteins into protein bodies (Chrispeels, Annu. Rev. Plant Physiol. Plant Mol. Biol., 42:21-53 (1991); Herman and Larkins, Plant Cell, 11:601-613 (1999)). These two pathways are illustrated in FIG. 1. In the ER lumen retention pathway (Path 1), the protein is synthesized on the surface of rough ER, and then translocated across the ER membrane into the lumen. Retention in the ER lumen can result in formation of protein bodies attached to or detached from the ER. In the Golgi body translocation pathway (Path 2), the translocated protein does not stay within the ER lumen, but is further translocated across the ER membrane and into the Golgi body. From there, the protein is released after its packaging into vacuoles to form vacuole-originated protein bodies. Protein modification, such as glycosylation, often occurs during this secretion process.

Numerous efforts have been made in the last two decades to identify determinants that direct proteins through the secretion pathways. Although a complete understanding has not been reached, various determinant peptides of storage proteins have been utilized in transgenic plants to target foreign proteins into designated cellular compartments, such as ER-originated protein bodies, vacuole-originated protein bodies, and apoplasts (Conrad and Fiedler, Plant Mol. Biol., 38:101-109 (1998); Moloney and Holbrook, Biotechnol. Genet. Eng. Rev, 14:321-336 (1997); Caimi et al., Plant Physiol. 110: 355-363 (1996); Boevink et al., Planta 208: 392-400 (1999)). However, few studies have focused on quantification of the translocated proteins' accumulation in these various plant tissues, a necessary requirement for the commercialization of plants as “protein production systems”. Among these studies, most show low or moderate levels of the translocation protein's accumulation. One of the exceptions to this generalization is the work of Ziegler et al. (Mol. Breed., 6:37-46 (2000)), which reports high yields of recombinant proteins (mean of 7.3% of total soluble protein with a high in one plant of 26% of total soluble protein) in leaves of Arabidopsis thaliana, using an apoplast-targeting cassette composed of the cauliflower mosaic virus 35S promoter, the tobacco mosaic virus Ω translational enhancer, the tobacco Pr1a signal peptide, and a nopaline synthase polyadenylation signal. However, many questions concerning the use of signal peptides to enhance accumulation of foreign proteins in different tissues have been raised due to a lack of quantitative and comparative data.

Thus, there remains a need for methods of accumulating proteins in high quantities in different plant tissues. Applicants have solved the stated problem by developing methods which enable high level accumulation of translocated proteins using sporamin signal peptide determinants and optionally the endoplasmic reticulum retention peptide. This has been accomplished by a detailed investigation of various combinations of signal peptide determinants, correlated with levels of accumulation of translocated protein accrued in specific tissues of the plant.

### SUMMARY OF THE INVENTION

The invention addresses the problem of accumulation of engineered proteins in plant tissues. The invention provides methods if increasing or enhancing the accumulation of proteins encoded by transgenes via the expression of translocation cassettes comprising elements which encodes a sporamin signal peptide, a sporamin pro-peptide or an endoplasmic reticulum retention peptide. Plant tissues expressing the cassettes of the invention demonstrated increased levels of proteins in the tissues expressing the cassettes.

Accordingly the invention provides a method for accumulating a translocated protein in a plant tissue comprising:

a) providing a plant having cells comprising a transgene comprising a protein translocation cassette encoding a protein having the general structure: SSP-TP-ER; wherein:

(i) SSP is a sporamin signal peptide;

(ii) TP is a protein to be translocated; and

(iii) ER is an endoplasmic reticulum retention peptide; and

b) growing the plant under conditions whereby the protein translocation cassette is expressed, and the translocated protein is accumulated in the plant tissues.

Similarly the invention provides a method for accumulating a translocated protein in a plant tissue comprising:

a) providing a plant having cells comprising a transgene comprising a protein translocation cassette encoding a protein having the general structure: SSP-TP; wherein:

(i) SSP is a sporamin signal peptide; and

(ii) TP is a protein to be translocated; and

b) growing the plant under conditions whereby the protein translocation cassette is expressed, and the translocated protein is accumulated in the plant tissues.

In an alternate embodiment the invention provides a method for accumulating a translocated protein in a plant tissue comprising:

a) providing a plant having cells comprising a transgene, the transgene comprising a protein translocation cassette encoding a protein having the general structure: SSP-SProP-TP; wherein:

(i) SSP is a sporamin signal peptide; and

(ii) TP is a protein to be translocated;

(iii) SProP is a sporamin pro-peptide; and

b) growing the plant under conditions whereby the protein translocation cassette is expressed, and the translocated protein is the plant tissues.

Additionally the invention provides a translocation protein cassette encoding a protein having the general structure: SSP-TP-ER; wherein:

(i) SSP is a sporamin signal peptide;

(ii) TP is a protein to be translocated; and

(iii) ER is an endoplasmic reticulum retention peptide selected from the group consisting of SEQ ID NO:33 and SEQ ID NO:34.

In similar fashion the invention provides a protein translocation cassette encoding a protein having the general structure: SSP-SProP-TP; wherein:

(i) SSP is a sporamin signal peptide;

(ii) TP is a protein to be translocated; and

(iii) SProP is a sporamin pro-peptide.

### BRIEF DESCRIPTION OF FIGURES AND SEQUENCE DESCRIPTIONS

FIG. 1 is an illustration of two protein secretion pathways directing formation of storage protein in protein bodies: Path 1 relates to ER lumen retention; Path 2 is involved in Golgi body translocation.

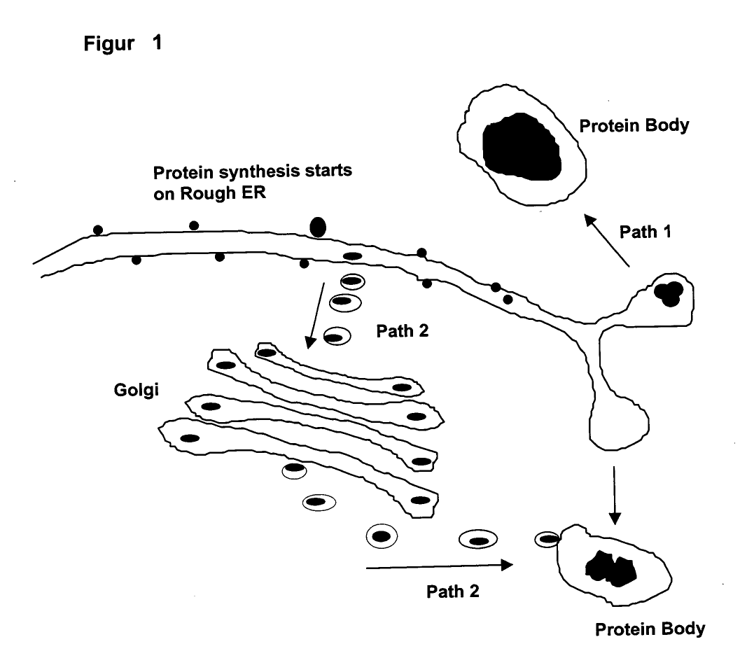

FIG. 2 shows the peptide sequence for a DP-1B monomer unit.

FIG. 3 is a summary of the DP-1B fusion protein designs.

FIG. 4A shows a plasmid map of master plasmid pGYV1/GUS, used for constitutive expression of transgenes. FIG. 4B diagrams the DP-1B-derived chimeric genes of plasmids pGYV501, pGYV502, and pGYV503, respectively.

FIG. 5A shows a plasmid map of master plasmid pGYV10/GUS, used for seed-specific expression of transgenes. FIG. 5B diagrams the DP-1B-derived chimeric genes of pGYV511, pGYV512, and pGYV513, respectively.

FIG. 6 shows results of immuno-blot assays used to detect constitutive expression of DP-1B fusion protein in leaves. FIG. 6A used DP-1B Abs as the primary antibody; FIG. 6B used Anti-His(C-term)-HRP as the primary antibody.

FIG. 7 shows a comparison of DP-1B fusion protein production yields in leaf tissues of transgenic Arabidopsis.

FIG. 8 shows results of immuno-blot assays used to detect seed-specific expression of DP-1B fusion proteins in seeds. FIG. 8A used DP-1B Abs as the primary antibody; FIG. 8B used Anti-His(C-term)-HRP as the primary antibody.

FIG. 9 is a comparison of DP-1B fusion protein production yields in seeds of transgenic Arabidopsis.

FIG. 10 presents results from PCR detection of DP-1B transgenes in progenies of the T1 transgenic plants.

FIG. 11 shows results of immuno-blot assays used to detect accumulation of DP-1B fusion proteins in progenies of transgenic plants. FIG. 11A examines T2 leaf protein extracts; FIG. 11B examines T3 seed protein extracts.

The following sequence descriptions and sequences listings attached hereto comply with the rules governing nucleotide and/or amino acid sequence disclosures in patent applications as set forth in 37 C.F.R. §1.821-1.825. The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NO:1 is the amino acid sequence of the sporamin signal peptide (SSP) (Hattori et al., Plant Mol. Biol., 5: 313-320 (1985)). This 21-amino acid determinant peptide sequence is responsible for translocation of sporamin into the ER lumen (Matsuoka and Nakamura, Proc. Natl. Acad. Sci. USA, 88: 834-838 (1991)).

SEQ ID NO:2 is the amino acid sequence of the sporamin pro-peptide (SProP), that is located downstream of the signal peptide (Hattori et al., Plant Mol. Biol., 5: 313-320 (1985)). This pro-peptide sequence enables transport of sporamin from the ER lumen to a protein body through the Golgi complex (Matsuoka and Nakamura, supra).

SEQ ID NO:3 is a spider silk variant derived from the amino acid sequence of the natural Protein 1 (Spidroin 1) of Nephila calvipes.

SEQ ID NO:4 and SEQ ID NO:5 are repeating peptides units frequently found in silk-like proteins.

SEQ ID NO:6 is the peptide sequence for a monomer unit of DP-1B SLP.

SEQ ID NO:7 is the highly conserved consensus motif found within a monomer unit of DP-1B.

SEQ ID NO:8 is the soft segment found within a monomer unit of DP-1B.

SEQ ID NO:9 is the hard segment found within a monomer unit of DP-1B.

SEQ ID NOs:10-14 are primers SPM+1, SPM+2, SPM+3, SPM−1, and SPM−2, respectively.

SEQ ID NOs:15 and 16 are the nucleotide and amino acid of the synthetically created sporamin targeting determinant coding region, including signal peptide and pro-peptide.

SEQ ID NOs:17, 18, and 19 are primers SPM-5′, SPM-S and SPM-V, respectively.

SEQ ID NO:20 represents an 83 bp nucleotide fragment encoding SSP suitable for DP-1B fusion protein construction.

SEQ ID NO:21 is the corresponding amino acid sequence for SSP.

SEQ ID NO:22 represents a 131 bp nucleotide fragment encoding SSP-SProP suitable for DP-1B fusion protein construction.

SEQ ID NO:23 is the corresponding amino acid sequence for SSP-SProP.

SEQ ID NOs:24 and 25 are primers H6 KDEL+ and H6 KDEL−, respectively.

SEQ ID NO:26 represents the amino acid sequence of the DNA adaptor encoding the H6 KDEL peptide. The corresponding nucleotide sequence is represented as SEQ ID NO:27 (top strand) and SEQ ID NO:28 (bottom strand).

SEQ ID NO:29 is a highly conserved sequence within all DP-1B fusion proteins.

SEQ ID NOs:30-32 are primers 35S—F, BC-F, and SPM-R, respectively.

SEQ ID NOs:33 and 34 are the ER retention peptides “KDEL” and “HDEL”, respectively.

### DETAILED DESCRIPTION OF THE INVENTION

The present invention provides methods for the accumulation of translocated proteins in plant tissues. The methods proceed by providing a plant with a protein translocation cassette that is a DNA construct comprising a sporamin signal DNA sequence, a coding region from a target gene, and optionally, a DNA sequence encoding a sporamin propeptide and/or an ER retention signal, according to the specific tissue of the plant where the translocated protein is to be expressed. More specifically:

1. To achieve accumulation of the translocated protein that is highest in the seed and high in the leaf of a plant, a translocation cassette that encodes a protein having the structure SSP-TP-KDEL, wherein SSP is the sporamin signal peptide, TP is a translocation protein and KDEL is the amino acid sequence KDEL (SEQ ID NO:33), is expressed in the desired plant cells.

2. To achieve accumulation of the translocation protein that is highest in the leaf of a plant, a translocation cassette that encodes a protein having the structure SSP-TP, wherein SSP is the sporamin signal peptide and TP is a protein to be translocated, is expressed in the desired plant cells.

3. To achieve accumulation of the translocated protein that is highest in the seed of a plant, a translocation cassette encoding a construct having the structure SSP-SProP-TP, wherein SSP is the sporamin signal peptide, SProP is the sporamin pro-peptide, and TP is a protein to be translocated, is expressed in the desired plant cells.

The work disclosed herein represents the first systematic determination of each respective targeting determinant peptide combination's capacity for translocated protein accumulation in leaf and seed tissue.

Abbreviations and Definitions

The following terms and definitions shall be used to fully understand the specification and claims.

“PCR” is the abbreviation for Polymerase Chain Reaction.

“TP” is the abbreviation for translocated protein.

“SSP” is the abbreviation for the sporamin signal peptide.

“SProP” is the abbreviation for the sporamin pro-peptide.

“KDEL” is the abbreviation for an ER retention peptide having the amino acid sequence “KDEL” (i.e., Lys Asp Glu Leu), represented as SEQ ID NO:33.

“SLP” is the abbreviation for silk-like protein.

“TSP” is the abbreviation for total soluble protein.

“Protein translocation” or “translocation” refers to the process of transporting a protein across a membrane. All proteins (except those made by the mitochondrial or chloroplast genomes) are synthesized on ribosomes located in the cytosol. Any proteins destined for sites other than the cytosol (i.e., inside an organelle or secretion from the cell) must be transported across at least one membrane in order to reach their final destinations.

All proteins that are translocated across the ER membrane are cotranslationally translocated, in that the protein is inserted across a membrane before the process of translation is completed. Thus, translation of the mRNA begins in the cytosol. As the nascent peptide emerges from the ribosome, its N-terminus contains a “signal sequence” that serves as a recognition sequence for a signal recognition particle (SRP). The SRP (a ribonucleoprotein) binds to the signal sequence and transports the nascent peptide, along with the ribosome/mRNA complex to which it is still attached, to the ER membrane. Translation is suspended during this process of transport. Once the ER membrane is reached, translation resumes and the emerging peptide is inserted through the ER membrane in an unfolded state. Other sequences in the peptide then provide signals for further localization of the protein.

A “signal peptide” (SP) is a short peptide sequence usually located at the amino terminus of a protein. These peptide sequences (typically 15-60 amino acids in length) target proteins from the cytosol into various plant organelles (e.g., the ER, mitochondria, chloroplasts, peroxisomes and nucleus) and are then cleaved from the mature protein following translocation. Examples of well known signal peptides include: PR1a tobacco SP (Hammond-Kosack, K. E., et al., PNAS, 91:10445-10449 (1994)), LeB4 SP (Legumin B4) from Vicia faba (Baumlein H., et al., Nucleic Acid Res, 14: 2707 1986), and tobacco PR-S SP (Cornelissen, B. J. C., et al., Nature 321:531-532 (1986)).

The term “sporamin signal peptide” (SSP) refers to the amino acid sequence of SEQ ID NO:1 and those sequences synthetically derived therefrom.

The term “sporamin pro-peptide” (SProP) refers to the amino acid sequence of SEQ ID NO:2 and those sequences synthetically derived therefrom.

“Accumulation” will hereinafter refer to a measurement or estimation of the amount of translocated protein at steady-state in a protein extract of plant cells, relative to the total soluble protein (TSP). This “steady-state” measurement of protein accumulation quantifies the amount or concentration of protein in terms of the amount of protein synthesized minus the amount lost by degradation processes. As will be apparent to one skilled in the art, accumulation will result only when the degradation of a protein occurs at a slower rate than the rate of protein synthesis. In like manner, accumulation will not occur when protein degradation occurs at a rate equal to or greater than the rate of protein synthesis. Accumulation will be quantitated as a “% of TSP”.

Typically, accumulation of DP-1B protein (“DP-1B accumulation”) will be determined in plant tissues in the disclosure herein. Preferred DP-1B accumulation in leaf when targeted to the ER lumen will be at least an average among a set of transformants of about 1.2% TSP, and more preferred DP-1B accumulation in leaf when targeted to the apoplast will be an average among a set of transformants of about 2.4% TSP or greater. In contrast, preferred seed DP-1B accumulation when targeted to the vacuole will be an average among a set of transformants of about 5.5% TSP, and more preferred accumulation when targeted to the ER lumen will be an average among a set of transformants of about 8.7% TSP or greater.

“Apoplast” refers to that region of the plant, which is outside the plasmamembrane system. Thus, the apoplast is comprises the non-living portion of the plant cell that includes the matrix of cell walls and the intercellular (free) spaces. Transport through the apoplast is “between the cells”, as compared to through the cytoplasm of the cells. In contrast to the apoplast, the symplast is that region of the plant, bounded by the plasma membrane, linking interconnected cells, via plasmodesmata.

“Targeting determinant peptide” refers to a signal peptide or an endoplasmic reticulum retention signal.

The term “protein translocation cassette” refers to a construct of DNA comprising one or more targeting determinant peptide coding sequences joined to a protein encoding sequence derived from a target gene such that the protein produced from the protein translocation cassette is to be translocated to the ER lumen, apoplast, or vacuole of a plant tissue.

A “target gene” refers to a gene that encodes a protein to be translocated through the addition of targeting determinant peptides.

A “translocated protein” refers to a protein, whose encoding sequence is derived from a target gene, and that is subjected to protein translocation when targeting determinant peptide sequences are adjoined to the target gene encoded protein.

The term “silk-like protein” will be abbreviated SLP and refers to natural silk proteins and their synthetic analogs having the following three criteria: 1.) the amino acid composition of the molecule is dominated by glycine and/or alanine; 2.) the consensus crystalline domain is arrayed repeatedly throughout the molecule; and 3.) the molecule is shear sensitive and can be spun into semicrystalline fiber. SLPs also include molecules that are modified variants of the natural silk proteins and their synthetic analogs defined above. An example of a SLP is “DP-1B”, which will hereinafter refer to any spider silk variant derived from the amino acid sequence of the natural Protein 1 (Spidroin 1) of Nephila calvipes as set forth in SEQ ID NO:3.

“DP-1B fusion protein” refers to the protein expressed from a protein translocation cassette containing the DP-1B coding region. DP-1B fusion protein refers to the unprocessed primary translated protein as well as to any processed derivatives of the primary protein.

“Monomers” are defined as those molecules that can undergo polymerization, thereby contributing discrete units to the essential structure of a polymer.

“Gene” refers to a nucleic acid fragment that expresses mRNA, functional RNA, or specific protein, including regulatory sequences. The term “native gene” refers to a gene as found in nature. The term “chimeric gene” refers to any gene that contains: 1.) DNA sequences, including regulatory and coding sequences, that are not found together in nature; or 2.) sequences encoding parts of proteins not naturally adjoined; or, 3.) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism or chimeric genes. The term “endogenous gene” refers to a native gene in its natural location in the genome of an organism. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In abbreviation of a chimeric gene, such as NOS::NPTII::OCS, it is understood that the 5′ most portion represents the promoter (NOS, for nopaline synthase promoter), and the 3′ most portion represents the 3′ terminator (OCS, for octapine synthase 3′ terminator).

“Foreign protein” refers to a protein that is not expressed from an endogenous gene of the plant. The foreign protein may be expressed from a transgene, a gene that is not stably maintained such as a gene that is part of Agrobacterium tumefaciens T-DNA, or from another type of introduced protein expression system such as an RNA viral vector.

“Synthetic genes” can be assembled from oligonucleotide building blocks that are chemically synthesized using procedures known to those skilled in the art. These building blocks are annealed and ligated to form gene segments that are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. “Plant preferred codons”, therefore, refers to the selection and use of preferred codons in plants. This bias can be targeted for either monocot or dicot plants, as necessary.

“Coding sequence” refers to a DNA or RNA sequence that codes for a specific amino acid sequence and excludes the non-coding sequences. The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters; however, some suitable regulatory sequences useful in the present invention will include, but are not limited to: constitutive plant promoters, plant tissue-specific promoters, plant developmental stage-specific promoters, inducible plant promoters and viral promoters.

The “3′ region” or “3′ terminator” means the 3′ non-coding regulatory sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence (e.g. for a target gene, etc.).

“Promoter” refers to a nucleotide sequence, usually upstream (5′) to a coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of an mRNA or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue-specificity of a promoter. It may be capable of operating in both orientations (normal or flipped), and of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

“Constitutive promoter” refers to promoters that direct gene expression in all tissues and at all times. “Regulated promoter” refers to promoters that direct gene expression not constitutively but in a temporally- and/or spatially-regulated manner and include tissue-specific, developmental stage-specific, and inducible promoters. The constitutive and regulated promoters include natural and synthetic sequences, as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered; numerous examples may be found in the compilation by Okamuro et al. (Biochemistry of Plants 15:1-82 (1989)). Since in most cases the exact boundaries of regulatory sequences have not been completely defined, DNA fragments of different lengths may have identical promoter activity. Typical regulated promoters useful in plants include, but are not limited to: safener-inducible promoters, promoters derived from the tetracycline-inducible system, promoters derived from salicylate-inducible systems, promoters derived from alcohol-inducible systems, promoters derived from the glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (e.g., leaves, shoot apical meristem, flower, or seeds), specific tissues (e.g., embryo or cotyledon), or specific cell types (e.g., leaf parenchyma, pollen, egg cell, microspore- or megaspore mother cells, or seed storage cells). These also include “developmental stage-specific promoters” that are temporally regulated, such as in early or late embryogenesis, during fruit ripening in developing seeds or fruit, in fully differentiated leaf, or at the onset of senescence. It is understood that the developmental specificity of the activation of a promoter and, hence, of the expression of the coding sequence under its control, in a transgene may be altered with respect to its endogenous expression. For example, when a transgene is under the control of a floral promoter, even when it is the same species from which the promoter was isolated, the expression specificity of the transgene will vary in different transgenic lines due to its insertion in different locations of the chromosomes.

“Plant developmental stage-specific promoter” refers to a promoter that is expressed not constitutively but at a specific plant developmental stage(s). Plant development goes through different stages; and in context of this invention, the germline goes through different developmental stages starting, say, from fertilization through development of embryo, vegetative shoot apical meristem, floral shoot apical meristem, anther and pistil primordia, anther and pistil, micro- and macrospore mother cells, and macrospore (egg) and microspore (pollen).

“Inducible promoter” refers to those regulated promoters that can be turned on in one or more cell types by a stimulus external to the plant, such as a chemical, light, hormone, stress, or a pathogen.

“Promoter activation” means that the promoter has become activated (or turned “on”) so that it functions to drive the expression of a downstream genetic element. Constitutive promoters are continually activated. A regulated promoter may be activated by virtue of its responsiveness to various external stimuli (inducible promoter), or developmental signals during plant growth and differentiation, such as tissue specificity (floral specific, anther specific, pollen specific, seed specific, etc.) and development-stage specificity (vegetative or floral shoot apical meristem-specific, etc.). In contrast, “conditionally activating” refers to activating a transgenic protein that is normally not expressed.

“Operably-linked” refers to the association of nucleic acid sequences on a single nucleic acid fragment so that the function of one is affected by the other. For example, a promoter is operably-linked with a protein-coding sequence or functional RNA-producing sequence when it is capable of affecting the expression of that associated sequence (i.e., the coding sequence or functional RNA-producing sequence is under the transcriptional control of the promoter). Coding sequences can be operably-linked to regulatory sequences in a sense or antisense orientation. “Unlinked” means that the associated genetic elements are not closely associated with one another and function of one does not affect the other.

“Expression” refers to the transcription and stable accumulation of sense (mRNA) or functional RNA. Expression may also refer to the production of active protein. “Over-expression” refers to a level of expression in transgenic organisms that exceeds levels of expression in normal or untransformed organisms. “Altered levels” refers to a level of expression in transgenic organisms that differs from that of normal or untransformed organisms

“Constitutive expression” refers to expression using a constitutive promoter. “Conditional” and “regulated expression” refer to expression controlled by a regulated promoter. “Transient” expression in the context of this invention refers to expression only in specific developmental stages or tissue in one or two generations. Finally, “non-specific expression” refers to constitutive expression or low level, basal (‘leaky’) expression in nondesired cells, tissues, or generations.

“Mature” protein or “active” protein refers to a polypeptide that has undergone post-translational processing. The mature or active protein no longer has any pre- or propeptides present, as these are removed from the primary translation product.

The term “altered plant trait” means any phenotypic or genotypic change in a transgenic plant relative to the wildtype or non-transgenic plant host.

“Transformation” refers to the transfer of a foreign gene into the genome of a host organism. Examples of methods of plant transformation include Agrobacterium-mediated transformation (De Blaere et al., Meth. Enzymol. 143:277 (1987)) and particle-accelerated or “gene gun” transformation technology (Klein et al., Nature (London) 327:70-73 (1987); U.S. Pat. No. 4,945,050). The terms “transformed”, “transformant” and “transgenic” refer to plants, plant tissues or calli that have been through the transformation process and contain a foreign gene integrated into their chromosome. The term “untransformed” refers to normal plants that have not been through the transformation process.

“Stably transformed” refers to cells that have been selected and regenerated on a selection media following transformation.

“Genetically stable” and “heritable” refer to chromosomally-integrated genetic elements that are stably maintained in the plant and stably inherited by progeny through successive generations.

“Wild-type” refers to the normal gene, virus, or organism found in nature without any known mutation.

“Genome” refers to the complete genetic material of an organism.

“Genetic trait” means a genetically determined characteristic or condition, which is transmitted from one generation to another.

“Primary transformant” refers to transgenic plants that are of the same genetic generation as the tissue which was initially transformed (i.e., not having gone through meiosis and fertilization since transformation). Thus, primary transformants usually refer to the “T0 generation”. But, in flower transformation, “primary transformant” refers to the T1 generation instead, because the transformants can only be identified from the T1 generation of plants.

A “set of transformants” is a group of two or more transformants derived from treatment with a single transformation vector. It is generally know by those skilled in the art that expression of a transgene in independent transformants varies due to the position of integration within the genome and other uncontrolled factors. Thus there will be individual transformants with higher and lower levels of transgene expression within a set of transformants.

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “plasmid” and “vector” refer to an extra chromosomal element often carrying genes which are not part of the central metabolism of the cell as well as other DNA segments, and usually in the form of circular double-stranded DNA molecules. Such DNA segments may include sequences directing autonomous replication, genome integrating sequences, and phage sequences. Further, a vector may be linear or circular, of a single- or double-stranded DNA or RNA, derived from any source, in which a number of nucleotide sequences have been joined or recombined into a unique construction. A DNA vector is capable of introducing a promoter fragment and DNA sequence for a selected gene product along with appropriate 3′ untranslated sequence into a cell. Typically, a DNA “vector” is a modified plasmid that contains: 1.) additional multiple restriction sites for cloning; and, 2.) achimeric gene that contains a DNA sequence encoding a selected gene product for expression in the host cell. This chimeric gene typically includes a 5′ promoter region, an ORF, and a 3′ terminator region, with all necessary regulatory sequences required for transcription and translation of the ORF. Thus, integration of the chimeric gene into the host results in a transgene that permits expression of the ORF in the chimeric gene.

As used herein the following abbreviations will be used to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Standard recombinant DNA and molecular cloning techniques used herein are well known in the art and are described by Sambrook, J., Fritsch, E. F. and Maniatis, T., Molecular Cloning: A Laboratory Manual, Second Ed., Cold Spring Harbor Laboratory Press, Cold Spring Harbor, N.Y. (1989) (hereinafter “Maniatis”); by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987).

Protein Translocation Cassettes

The present invention provides methods for the accumulation of translocated proteins that are targeted to the apoplast, ER lumen or vacuole in tissues of plants. The methods proceed by providing a plant with a protein translocation cassette having a DNA construct comprising a targeting determinant peptide coding sequence and a coding region from a target gene. The targeting determinant peptides include a signal peptide, a propeptide, and/or an ER retention peptide, according to the specific sub-cellular location in the plant tissue to which the translocated protein is targeted upon its synthesis (apoplast, ER lumen, or vacuole). Judicious choice of the regulatory elements that drive expression of the protein translocation cassette enables expression of the target gene to occur in a constitutive or regulated manner (e.g., seed-specifically).

Sporamin Signal Peptide and Sporamin Pro-Peptide

Sporamin accounts for about 80% of the total soluble protein in mature tuberous roots of the sweet potato Ipomoea batatas (Hattori et al., Plant Mol. Biol., 5: 313-320 (1985)). This group of storage proteins (apparent molecular weight of 25,000) lacks glycans and accumulates in the vacuoles of sweet potato roots. Following the identification of the sporamin sequence (Hattori et al., supra), Matsuoka et al. (J. Biol. Chem. 265(32): 19750-19757 (1990)) reported vacuolar targeting and post-translational processing of the precursor to sporamin in heterologous plant cells.

Work by Matsuoka and Nakamura (Proc. Natl. Acad. Sci. USA, 88: 834-838 (1991)) identified two tandem linked determinant peptide sequences at the N-terminus of sporamin as being critical for localization of sporamin. The first determinant peptide sequence is a 21-amino acid signal peptide (MKAFTLALFLALSLYLLPNPA) (SEQ ID NO:1), which translocates sporamin into the ER lumen. The second determinant sequence, which is downstream of the first determinant sequence, is a 16-amino acid propeptide (HSRFNPIRLPTTHEPA) (SEQ ID NO:2) that enables movement of sporamin from the ER lumen to a protein body through the Golgi complex. Although the sporamin signal peptide has since been used in numerous studies to target proteins to the ER lumen (see, for example, Caimi et al., Plant Physiol. 110: 355-363 (1996); Boevink et al., Planta 208: 392-400 (1999)), to date the advantages of using the sporamin propeptide in conjunction with the sporamin signal sequence for engineered protein translocation in plant hosts has not been realized.

Translocated Proteins

Translocated proteins of the present invention, encoded by target genes, will be those that convey a desirable phenotype on the transformed plant, those that encode markers useful in selection and breeding, or those that encode a desired protein product. Particularly useful target genes will include, but not be limited to: genes conveying a specific phenotype on a plant or plant cell, genes encoding a transformation marker, genes encoding a morphological trait, and genes encoding protein polymers and enzymes.

Target genes can encode proteins that are, for example, enzymes for primary or secondary metabolism in plants, proteins that confer disease or herbicide resistance, commercially useful non-plant enzymes, and proteins with desired properties useful in animal feed or human food. Additionally, foreign proteins encoded by the target genes will include seed storage proteins with improved nutritional properties, such as the high-sulfur 10 kD corn seed protein or high-sulfur zein proteins. Additional examples of target genes suitable for use in the present invention include: genes for disease resistance (e.g., gene for endotoxin of Bacillus thuringiensis, WO 92/20802) and herbicide resistance (mutant acetolactate synthase gene, WO 92/08794); seed storage protein genes (e.g., glutelin gene, WO 93/18643); genes for fatty acid synthesis (e.g., acyl-ACP thioesterase gene, WO 92/20236); genes for cell wall hydrolysis (e.g., polygalacturonase gene; see D. Grierson et al., Nucl. Acids Res., 14:8595 (1986)); and genes for anthocyanin biosynthesis (e.g., chalcone synthase gene; see H. J. Reif et al., Mol. Gen. Genet., 199:208 (1985)), ethylene biosynthesis (e.g., ACC oxidase gene; see A. Slater et al., Plant Mol. Biol., 5:137 (1985)), active oxygen-scavenging system genes (e.g., glutathione reductase gene; see S. Greer & R. N. Perham, Biochemistry, 25:2736 (1986)), and lignin biosynthesis genes (e.g., phenylalanine ammonia-lyase gene, cinnamyl alcohol dehydrogenase gene, o-methyltransferase gene, cinnamate 4-hydroxylase gene, 4-coumarate-CoA ligase gene, cinnamoyl CoA reductase gene; see A. M. Boudet et al., New Phytol., 129:203 (1995)).

Target genes may function as transformation markers. Transformation markers include selectable genes, such as antibiotic or herbicide resistance genes, which are used to select transformed cells in tissue culture, non-destructive screenable reporters (e.g., green fluorescent and luciferase genes), or a morphological marker (e.g., “shooty”, “rooty”, or “tumorous” phenotypes). Morphological transformation marker genes include cytokinin biosynthetic genes, such as the bacterial gene encoding isopentenyl transferase (IPT) (proposed as a marker for transformation by Ebumina et al. [Proc. Natl. Acad. Sci. USA 94:2117-2121 (1997)] and Kunkel et al. [Nat Biotechnol. 17(9): 916-919 (1999)]). Other morphological markers include developmental genes that can induce ectopic shoots, such as Arabidopsis STM, KNAT 1, or AINTEGUMANTA, Lec 1, Brassica “Babyboom” gene, rice OSH1 gene, or maize Knotted (Kn1) genes. Yet other morphological markers are the wild type T-DNA of Ti and Ri plasmids of Agrobacterium that induce tumors or hairy roots, respectively, or their constituent T-DNA genes for distinct morphological phenotypes, such as the shooty (e.g., cytokinin biosynthesis gene) or rooty phenotype (e.g. rol C gene). Use of a morphological transformation marker allows identification of a transformed tissue/organ and its subsequent removal (leaving behind the marker transgene) restores normal morphology and development to transgenic tissues. This is especially useful for in planta transformation, where the morphological marker is used to obtain abnormal transgenic organs that are then corrected by site-specific recombination to form morphologically and developmentally normal transgenic plants without going through the time and labor intensive tissue culture methods for transformation. Most preferably, the target gene by which the translocated protein used in the present invention is encoded can be a gene encoding a polymer protein. Many natural protein polymers, such as silk, collagen, and elastin are widely used for various purposes due to their unique mechanical properties and functionalities. Thus, in one embodiment, a preferred translocated protein is one encoded by a silk or SLP gene. These target genes may be naturally occurring or synthetic, and will generally be derived from silk producing organisms such as insects in the order Lepidoptera (including Bombyx mori and Nephila clavipes). Coding sequences for the silk or SLP polypeptides will generally be at least about 900 nucleotides in length, usually at least 1200 nucleotides in length, preferably at least 1500 nucleotides in length. Of particular interest are polypeptides which have as a repeating unit SGAGAG (SEQ ID NO:4) and GAGAGS (SEQ ID NO:5). Especially preferred SLPs are those described in WO 01/90389, the disclosure of which is herein incorporated by reference.

In one preferred embodiment, the silk or SLP may be derived from spider silk. There are a variety of spider silks that may be suitable for expression in plants. Many of these are derived from the orb-weaving spiders such as those belonging to the genus Nephila. Silks from these spiders may be divided into major ampullate, minor ampullate, and flagelliform silks, each having different physical properties. For a review of suitable spider silks, for example, see Hayashi et al. (Int J. Biol. Macromol. 24(2, 3): 271-275 (1999)). Those silks of the major ampullate are the most completely characterized and are often referred to as spider dragline silks. Natural spider dragline silk consists of two different proteins that are co-spun from the spider's major ampullate gland. The amino acid sequence of both dragline proteins has been disclosed by Xu et al. (Proc. Natl, Acad. Sci. U.S.A., 87:7120 (1990)) and Hinman and Lewis (J. Biol. Chem. 267: 19320 (1992)), and will be identified hereinafter as Dragline Protein 1 (DP-1) and Dragline Protein 2 (DP-2). Additionally, synthetic analogs of DP-1 have been designed that mimic both the repeating consensus sequence of the natural protein and the pattern of variation among individual repeats (WO 01/90389).

KDEL, an ER Retention Peptide

Since its discovery in 1992 (Denecke, J. et al., EMBO 11:2345-2355; Napier et al., J. Cell Sci. 102:261-271), the peptide sequences “KDEL” (SEQ ID NO:33) and “HDEL” (SEQ ID NO:34) have been universally recognized as signals for protein retention in the endoplasmic reticulum (ER).

Regulation of Protein Translocation Cassette Expression via Promoters

The present invention makes use of a variety of plant promoters to drive the expression of the protein translocation cassettes of the invention. Any promoter functional in a plant will be suitable including, but not limited to: constitutive plant promoters, plant tissue-specific promoters, plant development-specific promoters, inducible plant promoters, and flower-specific promoters. Regulated expression of each protein translocation cassette is possible by placing the protein translocation cassette under the control of a promoter that may be conditionally regulated.

Commonly used constitutive promoters in plants include the Arabidopsis SAMS (Mordhorst, A. P. et al. Genetics. 149(2):549-63 (1998)), Arabidopsis UBQ (ubiquitin) (Sun, C. K., and Callis, J. Plant 11 (5):1017-27 (1997)), CaMV 35S, Ti Plasmid OCS (octopine synthase), and Ti plasmid NOS (nopaline synthase) promoters.

Many tissue-specific and/or development-specific regulated genes and/or promoters have been reported in plants. These include genes encoding: the seed storage proteins (e.g., napin, cruciferin, β-conglycinin [cotyledon specific from soy], and phaseolin [cotyledon-specific from common bean]); zein or oil body proteins (e.g., the endosperm-specific maize zein and the embryo-specific Brassica oleosin) or genes involved in fatty acid biosynthesis (e.g., acyl carrier protein, stearoyl-ACP desaturase, and fatty acid desaturases (fad 2-1)); and other genes expressed during embryo development (e.g., Bce4 [EP 255378; Kridl et al., Seed Science Research 1:209-219 (1991)]). Particularly useful for seed-specific expression is the pea vicilin promoter (Czako et al., Mol. Gen. Genet. 235(1): 33-40 (1992)).

Other useful promoters for expression in mature leaves are those that are switched on at the onset of senescence, such as the SAG promoter from Arabidopsis (Gan et al., Science 270(5244): 1986-8 (1995)). Root or tuber specific promoters are also known, such as tobacco TobRB7, wheat lamda pox1 (peroxidase), and potato patatin B33. Flower or “floral”-specific promoters are those whose expression occurs in the flower or flower primordia (e.g., petunia chsA (chalcone synthase)). Anther-specific promoters (e.g., Arabidopsis A9 for tapetum-specific) and pollen-specific promoters (e.g., maize Pex1 [pollen extensin-like protein]; tomato Lat52 (Twell et al. Trends in Plant Sciences 3:305 [1998])) have also been identified and will be useful in the present invention. Recently, cDNA clones representing genes apparently involved in tomato pollen (McCormick et al., Tomato Biotechnology (1987) Alan R. Liss, Inc., New York) and pistil (Gasser et al., Plant Cell 1:15-24 (1989)) interactions have also been isolated and characterized.

A class of fruit-specific promoters expressed at or during anthesis through fruit development, at least until the beginning of ripening, is discussed in U.S. Pat. No. 4,943,674, the disclosure of which is hereby incorporated by reference. cDNA clones that are preferentially expressed in cotton fiber have been isolated (John et al., Proc. Natl. Acad. Sci. U.S.A. 89(13): 5769-73 (1992)). cDNA clones from tomato displaying differential expression during fruit development have been isolated and characterized (Mansson et al., Mol. Gen. Genet. 200:356-361 (1985); Slater et al., Plant Mol. Biol. 5:137-147 (1985)). The promoter for the polygalacturonase gene is active in fruit ripening. The polygalacturonase gene is described in U.S. Pat. No. 4,535,060, U.S. Pat. No. 4,769,061, U.S. Pat. No. 4,801,590, and U.S. Pat. No. 5,107,065, which disclosures are incorporated herein by reference.

Mature plastid mRNA for psbA (one of the components of photosystem II) reaches its highest level late in fruit development, in contrast to plastid mRNAs for other components of photosystem I and II which decline to nondetectable levels in chromoplasts after the onset of ripening (Piechulla et al., Plant Mol. Biol. 7:367-376 (1986)). A second promoter identified to function efficiently in chloroplasts is the tobacco Prrn promoter, a plastid rRNA operon promoter. In like manner, mitochondria promoters are also known, such as the wheat cox2 (cytochrome oxidase subunit 2) and soy atp9 (ATP snythase subunit 9) promoters. Other examples of tissue-specific promoters include those that direct expression in leaf cells following damage to the leaf (e.g., from chewing insects), in tubers (e.g., patatin gene promoter), and in fiber cells (e.g., the E6 developmentally-regulated fiber cell protein (John et al., Proc. Natl. Acad. Sci. U.S.A. 89(13):5769-73 (1992))). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., Plant Cell, 9:1527-1545 (1997)). Other tissue-specific promoters can be isolated by one skilled in the art (see U.S. Pat. No. 5,589,379).

Similarly, several inducible promoters (“gene switches”) have been reported. Many are described in the reviews by Gatz (Current Opinion in Biotechnology, 7:168-172 (1996); also, C. Annu. Rev. Plant Physiol. Plant Mol. Biol. 48: 89-108 (1997)). These include: the tetracycline repressor system; the Lac repressor system; copper-inducible systems (e.g., yeast acel); salicylate-inducible systems (e.g., the PR1a system); and glucocorticoid-(Aoyama T. et al., N-H Plant Journal 11:605-612 (1997)), estradiol-(e.g., “XVE”), and ecdysome-inducible systems. Also, included are the benzene sulphonamide-(U.S. Pat. No. 5,364,780) and alcohol (WO 97/06269 and WO 97/06268) inducible systems and glutathione S-transferase promoters. Other studies have focused on genes inducibly regulated in response to environmental stress or stimuli such as increased salinity, drought, pathogen attack, and wounding (Graham et al., J. Biol. Chem. 260:6555-6560 (1985); Graham et al., J. Biol. Chem. 260:6561-6554 (1985); Smith et al., Planta 168:94-100 (1986)). Specific promoters include the wound/pathogen inducible Asparagua officinalis AoPR1 and tomato PI-1 (proteinase inhibitor-1) promoters, and the water-stress inducible tobacco osmotin and rice rab-16A promoters. Accumulation of a metallocarboxypeptidase-inhibitor protein has been reported in leaves of wounded potato plants (Graham et al., Biochem Biophys Res Comm 101:1164-1170 (1981)). Other plant genes have been reported to be induced by methyl jasmonate, elicitors, heat-shock (e.g., Arabidopsis HSP18.2, soy Gmbsp17-E), anerobic stress, and herbicide safeners (e.g., maize In2-2).

Plant Hosts and Transformation Methods

The present invention additionally provides plant hosts for transformation with the present protein translocation cassettes. Moreover, the host plants for use in the present invention are not particularly limited. Examples of useful host plants are categorized as food plants (annuals), non-food plants (annuals), arboreous plants, and aquatic plants. Specific examples for each type of useful host plant are listed below.

Food plants (annuals): asparagus (Asparagus), banana (Musa), barley (Hordeum), blueberry (Vaccinium), broad bean (Vicia), cacao (Theobroma), capsicum pepper (Capsicum), carrot (Daucus), cassava (Manihot), corn (Zea), cucumber (Cucumis), eggplant (Solanum), lentil (Lens), lettuce (Lactuca), mango (Mangifera), oilseed, rape, canola, cabbage, broccoli, cauliflower (Brassica), oat (Avena), onions (Allium), papaya (Carica), peas (Pisum), peanut (Arachis), pineapple (Ananas), pinto bean, mung bean, lima bean (Phaseolus), potato (Solanum), pumpkin, zucchini (Cucurbita), radish (Raphanus), rice (Oryza), rye (Secale), sesame (Sesame), spinach (Spinaceae), sorghum (Sorghum), soybean (Glycine), strawberry (Fragaria), sugarcane (Saccharum), sugar beet (Beta), sunflower (Helianthus), sweet potato (Ipomoea), tomato (Lycopersicom), watermelon (Citrullus), wheat (Triticum), and yam (Dioscorea). Non-food plants (annuals): alfalfa (Medicago), amaranth (Amaranthus), angelica (Agelica), arabidopsis (Arabidopsis), castorbean (Ricinus), cotton (Gossypium), colewort (Crambe), dandelion (Taraxacum), flax (Linum), hemp (Cannabis), jojoba (Simmondsia), jute (Corchorus), kenaf (Hibiscus), lupine (Lupinus), petunia (Petunia), plantain (Plantago), sisal (Agave), snapdragon (Antirrhinum), switch grass (Panicum), and tobacco (Nicotiana).

Arboreous plants: apple (Malus), acacia (Acacia), chestnut (Castanea), citrus (Citrus), coconut (Cocos), coffee (Coffea), cypress (Cupressus), eucalypti (Eucalyptus), grape (Vitis), hemlock (Tsuga), hickory (Carya), maple (Acer), oak (Quercus), pear (Pyrus), peach, plum, cherry (Prunus), pine (Pinus), poplar (Populus), rose (Rosa), spruce (Picea), and walnut (Juglans).

Aquatic plants: brown alga (Laminaria), duckweed (Lemna), green alga (Chlamydomonas), and red alga (Porphyra).

However, the host plants for use in the present invention are not limited thereto.

One skilled in the art recognizes that the expression level and regulation of a protein translocation cassette in a plant can vary significantly from line to line. Thus, one has to test a number of lines to find one with the desired expression level and regulation leading to translocated protein accumulation.

A variety of techniques are available and known to those skilled in the art for introduction of constructs into a plant cell host. These techniques include transformation with DNA employing A. tumefaciens or A. rhizogenes as the transforming agent, particle acceleration, electroporation, etc. (See for example, EP 295959 and EP 138341). It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al. Bio/Technology 3:241 (1985); Byrne et al. Plant Cell, Tissue and Organ Culture 8:3 (1987); Sukhapinda et al. Plant Mol. Biol. 8:209-216 (1987); Lorz et al. Mol. Gen. Genet. 199:178 (1985); Potrykus. Mol. Gen. Genet. 199:183 (1985); Park et al., J. Plant Biol. 38(4): 365-71 (1995); Hiei et al., Plant J. 6:271-282 (1994)). The use of T-DNA to transform plant cells has received extensive study and is amply described (“Arabidopsis Protocols”, In Methods in Molecular Biology Vol. 82; Martinez-Zapater, J. M., and Salinas, J., Eds.; Humana: Totowa, N.J. (1998); Plant Molecular Biology, A Laboratory Manual, Clark, M. S., Ed. Springer-Verlag: Berlin, Heidelberg (1997); and Methods in Plant Molecular Biology, A Laboratory Course Manual, Maliga, P., et al., Eds; Cold Spring Harbor Laboratory: Cold Spring Harbor, N.Y. (1995)). For introduction into plants, the protein translocation cassettes of the invention can be inserted into binary vectors as described in the Examples.

Other transformation methods are available to those skilled in the art, such as high-velocity ballistic bombardment with metal particles coated with the nucleic acid constructs (see Kline et al. Nature (London) 327:70 (1987); and U.S. Pat. No. 4,945,050), direct uptake of foreign DNA constructs (see EP 295959), or techniques of electroporation (see Fromm et al. Nature (London) 319:791 (1986)). Once transformed, the cells can be regenerated by those skilled in the art. Of particular relevance are the recently described methods to transform foreign genes into commercially important crops, such as rapeseed (see De Block et al. Plant Physiol. 91:694-701 (1989)), sunflower (Everett et al. Bio/Technology 5:1201 (1987)), soybean (McCabe et al. Bio/Technology 6:923 (1988); Hinchee et al. Bio/Technology 6:915 (1988); Chee et al. Plant Physiol. 91:1212-1218 (1989); Christou et al. Proc. Natl. Acad. Sci USA 86:7500-7504 (1989); EP 301749), rice (Hiei et al., Plant J. 6:271-282 (1994)), and corn (Gordon-Kamm et al. Plant Cell 2:603-618 (1990); Fromm et al. Biotechnology 8:833-839 (1990)).

Transgenic plant cells are then placed in an appropriate selective medium for selection of transgenic cells that are then grown to callus. Shoots are grown from callus and plantlets generated from the shoot by growing in rooting medium. The various cassettes normally will be joined to a marker for selection in plant cells. Conveniently, the marker may be resistance to a biocide (particularly an antibiotic such as kanamycin, G418, bleomycin, hygromycin, chloramphenicol, herbicide, or the like). The particular marker used will allow for selection of transformed cells as compared to cells lacking the DNA that has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region that is not native to the gene from which the transcription-initiation region is derived.

To confirm the presence of the target genes in transgenic cells and plants, a Southern blot analysis or PCR can be performed using methods known to those skilled in the art. Expression products of the target genes can be detected in any of a variety of ways, depending upon the nature of the product, and include Western blots and enzyme assays. One particularly useful way to quantitate protein expression in different plant tissues is by use of a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

Accumulation of Translocated Proteins

The present invention permits targeting of translocated proteins to various cellular compartments within a plant, according to the specific construction of the protein translocation cassette (i.e., targeting to apoplast, ER lumen, or vacuole). As one skilled in the art may hypothesize, not all cellular locations within different tissues of a plant are equivalent or equipped to permit accumulation of translocated proteins. Seeds have been suggested to be an “optimal system for easy storage of recombinant proteins” (Conrad and Fiedler, Plant Mol. Biol. 38:101-109 (1998)), but a detailed study in support of this statement is lacking. In contrast, Moloney and Holbrook (Biotechnol. Genet. Eng. Rev, 14:321-336 (1997)) suggested that secretion of proteins into the apoplast may by very advantageous for numerous reasons.

The work disclosed herein represents the first systematic determination of protein accumulation, when targeting to each respective cellular location (apoplast, ER lumen, vacuole) in different plant tissues is used. Preferred constitutive accumulation in the leaf using apoplast targeting will be at least about 2.4% TSP averaged among a set of transformants, and more preferred accumulation will be about 4.8% TSP. Most preferred is accumulation of at least about 8.5% TSP, as reported herein. Similarly, preferred constitutive accumulation in the leaf using ER lumen targeting will be at least about 1.2% TSP averaged among a set of transformants, and more preferred accumulation will be about 2.4% TSP. Most preferred is accumulation of at least about 6.7% TSP or greater.

Preferred seed-specific accumulation using ER lumen targeting will be at least about 8.7% TSP among a set of transformants, and more preferred accumulation will be about 14.1% TSP. Most preferred is accumulation of at least about 18.2% TSP, as reported herein, or greater. Similarly, preferred seed-specific accumulation using vacuole targeting will be at least about 5.5% TSP averaged among a set of transformants, and more preferred accumulation will be about 8.2% TSP or greater.

Recovery Methods for the Translocated Proteins

The translocated proteins of the present invention may be extracted and purified from the plant tissue by a variety of methods, well known to those in the art. The particular downstream processing steps (e.g., transportation, purification, and further protein processing) selected for application must be critically evaluated for efficiency, to reduce costs of commercial protein production and purification.

When the translocated protein is a SLP, the preferred method of recovery will involve removal of native plant proteins from homogenized plant tissue by lowering pH and heating, followed by ammonium sulfate fractionation. Briefly, total soluble proteins are extracted from the transgenic plants by homogenizing plant tissues such as seeds and leaves. Native plant proteins are removed by precipitation at pH 4.7 and then at 60° C. The resulting supernatant is then fractionated with ammonium sulfate at 40% saturation. The resulting protein will be on the order of 95% pure. Additional purification may be achieved with conventional gel or affinity chromatography.

### DESCRIPTION OF THE PREFERRED EMBODIMENTS

DP-1B SLP (Silk-Like Protein) is a product of the synthetic DP-1B gene, which mimics the highly repetitive sequence of spider dragline silk spidroin 1 (Fahnestock and Bedzyk, Appl. Microbiol. Biotechnol., 47:33-39 (1997); U.S. Pat. No. 6,268,169). Previously, the constitutive and seed-specific production of 8-mer (64 kD) and 16-mer (125 kD) DP-1B SLP in transgenic plants has been demonstrated (WO 01/90389). This previous work showed that the DP-1B target genes were: 1.) introduced and integrated into plant genomes through either Agrobacterium-mediated transformation or particle-gun bombardment; 2.) stable during plant development and heritable through sexual reproduction; and 3.) found to accumulate in cytoplasm with an average yield of about 1% of TSP (total soluble protein).

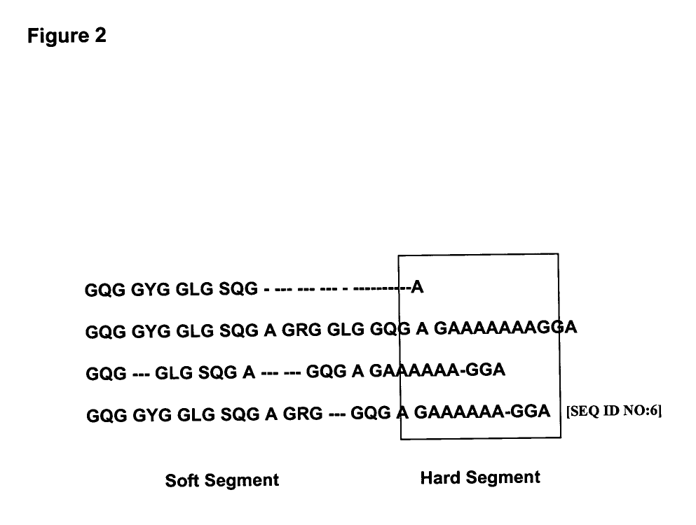

The peptide sequence for a monomer unit of DP-1B SLP is shown in FIG. 2. The 101 amino acid residues (SEQ ID NO:6) are aligned to reflect four repeats of the consensus motif. Each repeat includes a distinct amino acid deletion pattern, shown in dashes, to represent one of the naturally occurring patterns in spidroin 1. Additionally, the synthetic DP-1B SLP includes:

(1) An extremely alanine- and glycine-enriched amino acid composition;

(2) A highly repeated consensus motif of GQGGYGGLGSQGAGRGGLGGQGAGA₇GGA (SEQ ID NO:7);

(3) A soft segment of GQGGYGGLGSQGAGRGGLGGQG (SEQ ID NO:8); and

(4) A hard segment of AGA₇GGA within the motif (SEQ ID NO:9; shown as the boxed portion of FIG. 2).

These features determine the strong mechanical properties of DP-1B SLP. They also represent common structural signatures of many important natural structural proteins. Therefore, DP-1B SLP was deemed a useful target gene for the present investigation, aimed at developing a method for accumulation of proteins in high quantities in various cellular compartments of a plant system (e.g., the ER lumen, apoplast, or vacuoles). Methods so developed are expected to be applicable for production of many highly repetitive recombinant protein polymers, and other foreign proteins.

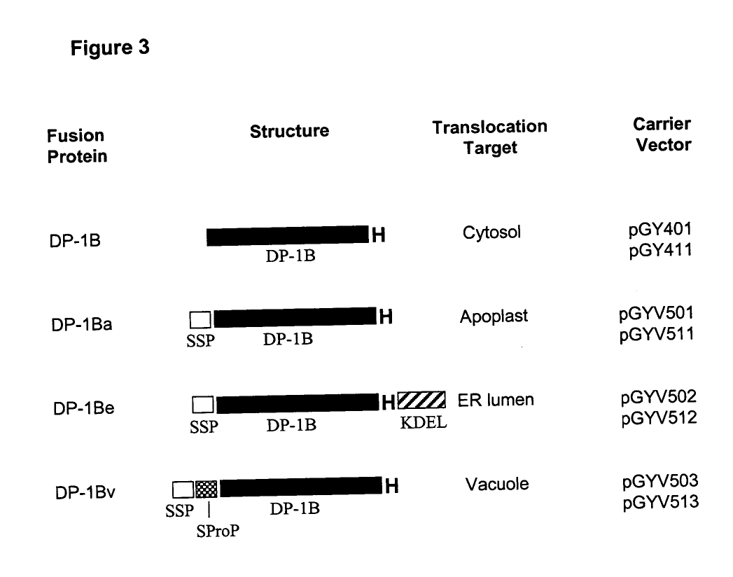

The examples herein targeted 8-mer DP-1B SLP (65 kD) to the apoplast, ER lumen, and vacuole of plant tissues utilizing appropriate combinations of targeting determinant peptides from sweet potato sporamin (i.e., the sporamin signal peptide and sproramin pro-peptide) and the KDEL ER retention peptide. A summary of the designs of these protein translocation cassettes comprising DP-1B fusion proteins is shown in FIG. 3. In the diagram the following symbols are utilized:

the black box represents an 8-mer “DP-1B SLP”;

“H” represents a C-terminal 6× Histidine tag;

the hatched box 3′ to the 6× Histine tag represents the ER retention peptide “KDEL” (SEQ ID NO:33);

the white box represents a sporamin signal peptide “SSP”; and

the checkered box represents a sporamin pro-peptide “SProP”.

As described in the column labeled “Target Compartment”, the 8-mer DP-1 B SLP was designed to accumulate in apoplast by fusing a sporamin signal peptide to the N-termius of DP-1B creating DP-1Ba. Vacuole accumulation was targeted by fusing a tandem array of the sporamin signal peptide and the propeptide to the N-termius of DP-1B creating DP-1 By. And, accumulation in the ER lumen was specified by fusing a sporamin signal peptide and a KDEL peptide to the N- and C-termini of DP-1B, respectively, creating DP-1Be.

The well defined 35S (CaMV 35S) promoter and soy BCα′ (β-conglycinin α prime sub-unit) promoter were operably linked to the protein translocation cassettes to drive strong constitutive and seed-specific expression, respectively. Arabidopsis thaliana was employed to carry and express the genes encoding 8-mer DP-1B protein translocation cassettes because it is a widely accepted model of flowering higher plants and it offers convenience in transformation, selection, growth, examination, and genetic crossing.

Among the approaches utilized to target DP-1B SLP into apoplast, ER lumen, or vacuoles of plant tissues, it was determined that:

1.) targeting to the apoplast and ER lumen greatly enhanced DP-1B SLP accumulation in leaves; and

2.) targeting to the ER lumen and vacuole greatly enhanced DP-1B SLP accumulation in seeds without disruption of protein quality.

Of these approaches, targeting to the apoplast led to the highest levels of accumulation of the translocated protein in leaves. Average accumulation (N=8) was 2.47% TSP. In contrast to the results obtained in leaves, targeting to the ER lumen led to the highest levels of DP-1B accumulation in seeds (average accumulation 8.74% TSP, where N=27). Many seeds achieved DP-1B SLP accumulation levels greater than 15% of TSP in pGYV512 transformants, with maximum accumulation measured as 18.2% of TSP. The accumulation of the translocated protein could be even greater than reported herein, if the small portion of the seed collection that had returned to the wild-type genotype (due to segregation) is considered. These seeds would produce no DP-1B SLP, and therefore reduce the accumulation average in the seed population. An additional advantage of the present method is the fact that the phenotype of protein accumulation is heritable in plant progenies.

The invention has provided the first evidence that sporomin targeting determinant peptides could greatly enhance foreign protein accumulation in plant tissues through the native protein targeting processes. Additionally, the invention provides the first example of high level accumulation of a highly repetitive recombinant protein in plants, when specifically targeted to cellular or extracellular compartments. Recorded levels of SLP production and accumulation approach those required for commercial production of these types of recombinant proteins, using a combination of the seed-specific expression and the ER lumen-targeted accumulation. In addition, seed-based production provides an efficient method for the storage, transportation, and processing of DP-1B SLP. Finally, it is expected that the methodology of the present invention based on use of sporamin determinant peptides and the ER retention peptide for specific protein targeting will be readily applicable to any foreign protein suitable for expression in a plant, and enable high level accumulation of these foreign protein products.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, N.Y. (1989) (Maniatis) and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, Eds., American Society for Microbiology, Washington, D.C. (1994)) or by Thomas D. Brock in Biotechnology: A Textbook of Industrial Microbiology, Second Ed., Sinauer Associates, Inc.: Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, MI), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used, the gap creation default value of 12 and the gap extension default value of 4 were used. Where the GCG “Gap” or “Bestfit” programs were used, the default gap creation penalty of 50 and the default gap extension penalty of 3 were used. In any case where GCG program parameters were not prompted for, in these or any other GCG program, default values were used.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” means micromole(s), “g” means gram(s), “μg” means microgram(s), “ng” means nanogram(s), “U” means unit(s), “bp” means base pair(s), “kB” means kilobase(s), and “kD” means kilodalton(s).

### Example 1

### Construction of DP-1B-derived Protein Translocation Cassettes with Protein Targeting Features

Example 1 describes: (1) the synthesis of coding sequences for the sporamin targeting determinant peptides (sporamin signal peptide and sporamin propeptide); (2) the synthesis of coding sequences for the ER retention peptide; and (3) the adjoining of these targeting determinant sequences to the DP-1B coding sequence to create three specific protein translocation cassettes targeted to the apoplast, ER lumen, or vacuole of a plant.

Synthesis of Coding Sequences for Sporamin Targeting Determinant Peptides

The coding sequences for sporamin signal peptide and propeptide were taken from Hattori et al. (Plant Mol. Biol., 5: 313-320 (1985); SEQ ID NOs:1 and 2). To prepare these sequences synthetically, five complementary and overlapping nucleotide oligomers of nucleotides were synthesized (SEQ ID NO:10-14). Oligomers were pooled into a 100 μL phosphorylation reaction, which contained 200 pmole of each oligomer, 0.1 mM ATP, 20 units T4 polynucleotide kinase (Life Technologies, Rockville, Md.), and 1× forward reaction buffer (Life Technologies). After a 0.5-hr incubation at 37° C., the reaction was stopped and cleaned up using Qiaquick Nucleotide Removal Kit (QIAGEN, Valencia, Calif.).

The phosphorylated oligomers were then subjected to an annealing program on a GeneAmp PCR System 9600 (Perkin Elmer, Norwalk, Conn.), which included heating at 98° C. for 10 min, followed by a 75° C. temperature drop at a slope of 1° C. per min. Finally, the annealed oligomers were ligated at 16° C. overnight in a 100 μL reaction containing 2 units T4 DNA ligase and 1× ligase reaction buffer (Life Technologies). The reactions were cleaned up using QIAquick PCR Purification Kit (QIAGEN). The resultant double-strand DNA sequence and its translated peptide sequence are presented as SEQ ID NO:15 and 16, respectively. These sequences were identical to the sweet potato sporamin targeting determinant peptide sequences for the upstream signal peptide and downstream pro-peptide, with the exception of an extra codon for glycine (GGG) added immediately following the start codon (ATG) to introduce a NcoI site.

To prepare the coding sequences for SSP (an individual sporamin signal peptide) and SSP-SProP (a tandem array of sporamin signal peptide and pro-peptide) suitable for integration with the DP-1B SLP gene, three nucleotide oligomers were synthesized as PCR primers (SEQ ID NO:17-19). Appropriate pairs of the primers were applied in a 50 μL-PCR reaction, containing 0.25 μM of each dNTP, 2.5 units Pfu DNA polymerase (STRATAGENE, La Jolla, Calif.), 1×Pfu buffer, 25 pmole of each primer, and 2 μL assembled DNA as template. The reactions were carried out on a GeneAmp PCR System 9600 for 30 cycles, following a program of: 30 sec denaturation at 94° C., 30 sec annealing at 55° C., and 1 min amplification at 72° C.

Primers SPM-5′ (SEQ ID NO:17) and SPM-s (SEQ ID NO:18) amplified a 83 bp nucleotide fragment encoding for SSP (SEQ ID NO:20; amino acid sequence shown as SEQ ID NO; 21), while primers SPM-5′ (SEQ ID NO:17) and SPM-v (SEQ ID NO:19) resulted in a 131 bp nucleotide fragment encoding SSP-SProP (SEQ ID NO:22; amino acid sequence shown as SEQ ID NO:23). Both PCR products contained one extra codon for glycine after the start codon and an extra nucleotide fragment before the start codon in order to introduce a NcoI site at the start codon. As shown below, they also contained: 1.) an alternative codon for the last alanine in SSP and SSP-SProP; and 2.) an additional downstream sequence to create a BgIII site.

SEQ ID NO:20 and 21

```
SEQ ID NO:20 and 21
            NcoI BgIII
CCACCGCCATGGGGAAAGCCTTCACACTCGCTCTCTTCTTAGCTCTTTCCCTCTATCTCCTGCCCAATCCAGCTAGATCTCAA
                M G K A F T L A L F L A  L S L Y L L P N P A R S Q
SEQ ID NO:22 and 23
            NcoI
        CCACCGCCATGGGGAAAGCCTTCACACTCGCTCTCTTCTTAGCTCTTTCCCTCTATCTCCTGCCCAA
                        M G K A F T L A L F L A L S L Y L L P N
                                                                                                              BgIII
        CCAGCCCATTCCAGGTTCAATCCCATCCGCCTCCCCACCACACACGAACCCGCTAGATCTCAA
              P A H S R F N P I R L P T T H E P A R S Q
```

Because this additional sequence actually encoded the amino acids RSQ, identical to the 5′ region of the DP-1B coding region, BgIII digestion permitted compatibility between SSP or SSP-SProP and the 5′ end of the DP-1B coding region.

Synthesis of Coding Sequences for the ER Retention Peptide

To prepare the coding sequence for a peptide containing a 6×-histidine tag and the KDEL ER retention signal (H6 KDEL), two oligomers were designed as h6 kdel+ (SEQ ID NO:24) and h6 kdel− (SEQ ID NO:25), according to the rules of plant codon bias (Murray, et al., Nucl. Acid. Res., 17: 477-498 (1989)). Both oligomers were mixed in a 20-μL annealing reaction containing 2.5 nmole of each oligomer and 1×TE buffer and subjected to an annealing program on a GeneAmp PCR System 9600 (Perkin Elmer). This included heating at 98° C. for 10 min followed by a 75° C. temperature drop at a slope of 1° C. per 5 min. The reaction resulted in a double-stranded DNA adaptor encoding a H6 KDEL peptide (SEQ ID NO:26; corresponding nucleotide sequences for the top and bottom strands shown below as SEQ ID NOs:27 and 28) with a stop codon at its 3′ end.

SEQ ID NOs:26-28

```
GATCCCATCACCATCACCATCACAAGGATGAGCTTTAAGGTAC
        GGTAGTGGTAGTGGTAGTGTTCCTACTCGAAATTC
    S H H H H H H K D E L
```

The adapter also introduced a 5′ sticky end compatible with the BamHI site at the end of DP-1B coding region (see details below) and a 3′ sticky end compatible with a KpnI site.

Adjoining of the Targeting Determinant Sequences to the DP-1B Coding Sequence

The 8-mer DP-1B coding sequence for plants was provided in plasmid pGY101, a pBluescript-based plasmid. Specifically, the polylinker region of the plasmid contained a synthetic 8-mer DP-1B gene with a C-terminal 6×-histidine tag (WO 01/90389).

DP-1B was modified for targeting to specific compartments of a plant tissue, according to the methodology that follows. First, pGY101 was linearized at the N-terminus of the DP-1B coding region with NcoI and BgIII enzymes in a standard digestion reaction. The reactions were cleaned up using a QIAquick PCR Purification Kit (QIAGEN). PCR amplified DNA fragments encoding SSP and SSP-SProP were digested and cleaned up using identical methods and each was inserted into linearized pGY101 in a standard DNA ligation reaction with T4 DNA ligase. Insertion of the SSP fragment resulted in plasmid pGYV101, which contained a protein translocation cassette encoding the fusion protein DP-1Ba. Insertion of the SSP-SProP fragment resulted in plasmid pGYV103, which contained a protein translocation cassette encoding the fusion protein DP-1Bv. Both pGYV101 and pGYV103 were prepared from STBL2 E. coli cells (Life Technologies) using QIAprep Spin Miniprep Kits (QIAGEN).

For creation of a fusion protein targeted to the ER lumen, pGYV101 was digested with BamHI, ClaI, and KpnI enzymes. This removed a BamHI-KpnI fragment encoding the 6×-histidine tag and stop codon at the 3′ end of the DP-1Ba coding region. In its place, the DNA adapter (SEQ ID NOs:27 and 28) encoding the H6 KDEL peptide with a stop codon at its end was ligated into the linearized pGYV101 between the BamHI and KpnI sites. The resultant plasmid was named pGYV102, and it contained the protein translocation cassette encoding fusion protein DP-1Be. Plasmid pGYV102 was also prepared from STBL2 E. coli cells.

Each of these constructs is summarized in Table 1 and graphically illustrated in FIG. 3. The newly integrated targeting determinant coding regions and their adjunction with the DP-1B coding sequence was confirmed directly by DNA sequencing.

TABLE 1
Summary of Intermediate Plasmids

| Plasmid | Parent Plasmid | Coding Sequence and Target |
|---|---|---|
| pGY101 | pBluescript SK(+) | DP-1B (with a 6x histidine tag), for |
| | | targeting to the cytosol |
| pGYV101 | pGY101 | DP-1Ba (with a 6x histidine tag), for |
| | | targeting to the apoplast |
| pGYV102 | pGYV101 | DP-1Be (with a 6x histidine tag), for |
| | | targeting to the ER lumen |
| pGYV103 | pGY101 | DP-1Bv (with a 6x histidine tag), for |
| | | targeting to the vacuole |

### Example 2

### Binary Vector Construction for Expression of DP-1B Fusion Proteins

Example 2 describes: (1) the preparation of two master binary vectors, pGYV1/GUS and pGYV10/GUS; (2) the construction of vectors for constitutive expression of DP-1B fusion proteins (from Example 1) in leaf tissue of plants; and (3) the construction of vectors for seed-specific expression of DP-1B fusion proteins (from Example 1).

Preparation of Master Binary Vectors pGYV1/GUS and pGYV10/GUS

Because each of the DP-1B-derived protein translocation cassettes described in Example 1 could be isolated from the host vector as a uniquely orientated NcoI-KpnI DNA fragment, it was useful to create several master expression vectors that would permit facile chimeric gene construction by NcoI/KpnI digestion and ligation.

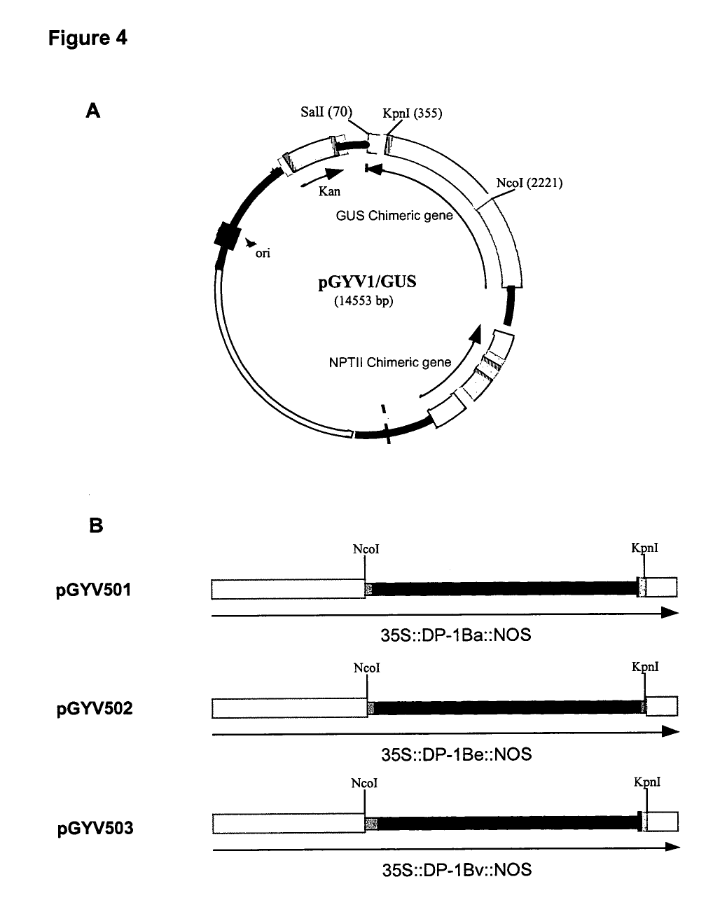

Master expression vector pGYV1/GUS (FIG. 4A) was derived from the binary vector pZBL1, with additional elements provided from plasmid pML63 (provided by DuPont Agricultural Products (Wilmington, Del.); described in WO 01/90389). Thus, the plasmid had two chimeric genes within its “T-DNA region”. The NPTII gene (NOS::NPTII::OCS; having the nopaline synthase promoter and octopine synthase 3′ terminator sequence) conferred a kanamycin-resistant phenotype for transformant selection. The 35S::GUS::NOS gene (having the CaMV 35S promoter and the nopaline synthase 3′ terminator sequence) led to constitutive expression of the GUS transgene. Because the NcoI site within the NPTII coding region had been eliminated in this vector, any coding region with a unique NcoI site at its start codon and a unique KpnI site downstream of its stop codon could be easily integrated into the chimeric gene to replace GUS.

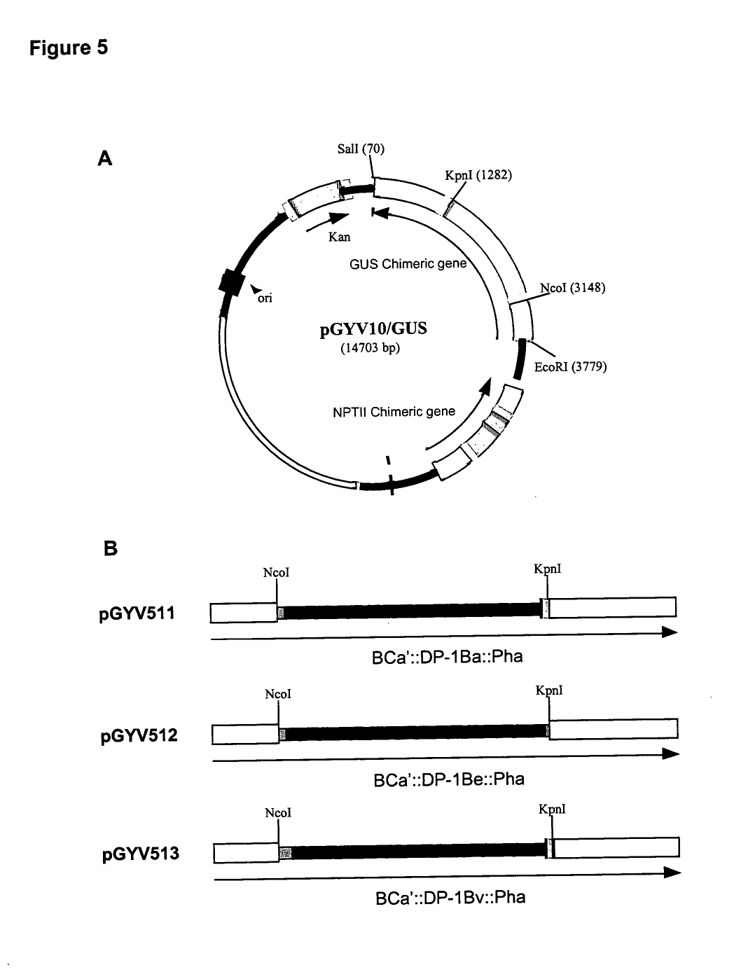

A second master expression vector was constructed and named pGYV10/GUS (FIG. 5A). This vector was also a pZBL1-derived binary vector with a structure very similar to pGYV1/GUS. However, the vector's chimeric gene was BCα′::GUS::Pha for seed-specific expression. The BCα′ promoter and Pha (phaseolin) 3′ terminator sequence were introduced into the vector from pGY213 (WO 01/90389). Like pGYV1/GUS, the GUS coding region of the chimeric gene in pGYV10/GUS could be replaced by any coding region with a unique NcoI site at its start codon and unique KpnI site downstream of its stop codon.

During construction of the master expression vectors, all deletions, insertions, and mutagenesis were confirmed directly by DNA sequencing. Both vectors were prepared from XL1-Blue E. coli cells (Stratagene) using QIAprep Spin Miniprep Kits (QIAGEN, Valencia, Calif.). These vectors are summarized in Table 2.

TABLE 2
A Summary of The Master Expression Vectors

| | | Parent | | |
|---|---|---|---|---|
| Name | FIGURE | Plasmid | Selection | Chimeric gene |
| pGYV1/ | 4A | pZBL1 | NOS::NPTII::OCS | 35S::GUS::NOS |
| GUS | | | | |
| pGYV10/ | 5A | pZBL1 | NOS::NPTII::OCS | BCα′::GUS::Pha |
| GUS | | | | |

Vector Construction for Constitutive Expression of DP-1B Fusion Proteins in Leaf Tissue:

Master expression vector pGYV1/GUS provided a backbone for several constitutive expression binary vectors. First, the backbone vector was digested with NcoI and KpnI in a standard digestion reaction. The GUS fragment was separated from the remainder of the vector on a TBE agarose gel and the vector fragment was purified using a QIAquick Gel Extraction Kit (QIAGEN). The DP-1Ba, DP-1Be, and DP-1Bv protein translocation cassettes were obtained from plasmids pGYV101, pGYV102, and pGYV103, respectively. Each plasmid was subjected to digestion reactions with NcoI, KpnI, and PvuI. NcoI and KpnI cleaved the protein translocation cassette DNA fragments from their carriers, while PvuI further digested the carrier sequence into smaller fragments that would be visually distinguishable from the cassette fragments. All protein translocation cassette DNA fragments were isolated by the gel-purification method described previously.

Finally, each protein translocation cassette DNA fragment was individually subcloned into the prepared vector backbone of pGYV1/GUS in a standard ligation reaction, which resulted in expression vectors pGYV501, pGYV502, and pGYV503. All expression vectors were prepared with STBL2 E. coli cells (Life Technologies) using QIAprep Spin Miniprep Kits (QIAGEN, Valencia, Calif.). These expression vectors are summarized in Table 3.

TABLE 3
Vectors for Constitutive Expression in Leaf Tissues*

| | Parent | | |
|---|---|---|---|
| Name | Plasmid | Selection | Chimeric gene |
| pGYV501 | pGYV1/GUS | NOS::NPTII::OCS | 35S::DP-1Ba::NOS |
| pGYV502 | pGYV1/GUS | NOS::NPTII::OCS | 35S::DP-1Be::NOS |
| pGYV503 | pGYV1/GUS | NOS::NPTII::OCS | 35S::DP-1Bv::NOS |

*Vectors are also illustrated in FIG. 4B.

Each vector possessed two chimeric genes in the T-DNA region, the sequence that integrates into the plant genome during Agrobacterium-mediated transformation. The NPTII chimeric gene provided a kanamycin-resistance selective marker. The generic 35S::modified-DP-1B::NOS chimeric gene leads to constitutive expression of the DP-1B transgenes and accumulation of the translocated protein in various tissue compartments, depending on the targeting determinant sequences in the vectors. Specifically, pGYV501 (containing DP-1Ba) was designed to accumulate in the apoplast, pGYV502 (containing DP-1Be) was designed to accumulate in the ER lumen, and pGYV503 (containing DP-1Bv) was designed to accumulate in the vacuole.

Vector Construction for Seed-Specific Expression of DP-1B Fusion Proteins

Master expression vector pGYV10/GUS provided a backbone for several seed-specific expression vectors. The vector fragment of pGYV10/GUS was separated from the GUS fragment and prepared as described above for pGYV1/GUS. The protein translocation cassettes DP-1 Ba, DP-1Be, and DP-1Bv (derived from plasmids pGYV101, pGYV102, and pGYV103, respectively) were prepared as described above and then subcloned into the vector fragment of pGYV10/GUS via standard ligation reactions. This resulted in seed-specific expression vectors pGYV511, pGYV512, and pGYV513 (Table 4). All expression vectors were prepared from STBL2 E. coli cells using QIAprep Spin Miniprep Kits.

TABLE 4
Vectors for Seed-Specific Expression*

| | Parent | | |
|---|---|---|---|
| Name | Plasmid | Selection | Chimeric gene |
| pGYV511 | pGYV10/GUS | NOS::NPTII::OCS | BCα′::DP-1Ba::Pha |
| pGYV512 | pGYV10/GUS | NOS::NPTII::OCS | BCα′::DP-1Be::Pha |
| pGYV513 | pGYV10/GUS | NOS::NPTII::OCS | BCα′::DP-1Bc::Pha |

*Vectors are also illustrated in FIG. 5B.

These seed-specific expression vectors were similar to the constitutive expression vectors; however, their DP-1B-derived chimeric genes had a BCα′ promoter and Pha 3′ terminator for seed-specific expression of the DP-1B-derived fusion proteins. DP-1B SLP translocated protein products were designed to accumulate in three compartments in seed tissues. More specifically, pGYV511 (containing DP-1Ba) targets accumulation to the apoplast, pGYV512 (containing DP-1Be) targets accumulation to the ER lumen, and pGYV513 (containing DP-1Bv) targets accumulation to the vacuole.

### Example 3

### Arabidopsis Transformation and Primary Transformant Selection

Example 3 describes: (1) the preparation of Agrobacterium strains carrying each of the expression vectors containing DP-1B protein translocation cassettes (from Example 2); (2) the transformation of Arabidopsis with these Agrobacterium strains; and (3) the selection of primary transformants.

### Preparation of Agrobacterium Strains Carrying DP-1B-Derived Chimeric Genes

To make competent Agrobacterium cells, a colony of Agrobacterium strain C58C1 (pMP90) (Koncz and Schell, Mol. Gen. Genet., 204: 383-396 (1986)) was grown to an OD₆₀₀ of 1.0 in 1 L YEP media, which includes 10 g Bacto peptone, 10 g yeast extract, and 5 g NaCl. The culture was chilled on ice and cells were collected by centrifugation. Cells were resuspended in ice-cold 20 mM CaCl₂ solution and stored at −80° C. in 0.1 mL aliquots.

A freeze-thaw method was used to introduce pGYV501, pGYV502, pGYV503, pGYV511, pGYV12, and pGYV513 into Agrobacterium. First, 1 μg plasmid DNA from each of these constructs was added to the frozen aliquot of Agrobacterium cells. The mixture was thawed at 37° C. for 5 min, diluted with 1 mL YEP medium, and then gently shaken at 28° C. for 2 hrs. Cells were collected by centrifugation, spread on a YEP agar plate containing 25 mg/L gentamycin and 50 mg/L kanamycin, and grown at 28° C. for 2 to 3 days. Agrobacterium transformants were confirmed by mini-preparation and restriction enzyme digestion of plasmid DNA by standard methods, except that lysozyme (Sigma, St. Louis, Mo.) was applied to the cell suspension prior to DNA preparation to enhance cell lysis.

Transformation of Arabidopsis

Arabidopsis thaliana was grown until bolt emergence in 3″ square pots of Metro Mix soil (Scotts-Sierra, Maryville, Ohio) at a density of 5 plants per pot. Growth occurred under a controlled temperature (22° C.) and an illumination cycle of 16 hr light/8 hr dark. Plants were decapitated 4 days before transformation. Agrobacteria carrying pGY501, pGY502, pGY503, pGY511, pGY512, and pGY513 plasmids were each grown in LB medium (1% bacto-tryptone, 0.5% bacto-yeast extract, 1% NaCl, pH 7.0) containing 25 mg/L gentamycin and 50 mg/L kanamycin at 28° C., until the culture reached an OD₆₀₀ value of 1.2. Cells were collected by centrifugation and resuspended in infiltration medium (½×MS salt, 1×B5 vitamins, 5% sucrose, 0.5 g/L MES, pH 5.7, 0.044 μM benzylaminopurine) to an OD₆₀₀ of approximately 0.8.

A vacuum infiltration method was employed to transfect Arabidopsis plants with Agrobacteria carrying the expression vectors described in Example 2. Briefly, a 500 mL Magenta Box was filled with an infiltration medium suspension of Agrobacteria and covered with a 3″ square pot containing 5 Arabidopsis plants in an inverted position, so that each plant was entirely submerged in the suspension. The assembly was placed in an Isotemp Vacuum Oven model 281 (Fisher Scientific, Pittsburgh, Pa.) and subjected to infiltration for 5 min under 30 mm Hg vacuum. At least 3 pots of plants were infiltrated with each Agrobacterium strain. They were then laid on their sides in a Saran wrap sealed flat and permitted to recover overnight at room temperature. The transfected Arabidopsis plants were grown to maturation under standard growth conditions (22° C., 16 hr light/8 hr dark). T1 seeds were collected from plants in each pot, dried for one week, and stored at room temperature. Primary transformants were included in these T1 seed collections.

Selection of Primary Transformants

To select primary transformants, T1 seeds were sterilized in 1 mL of 50% bleach and 0.02% Triton X-100 solution for 7 min, followed by 5 rinses in sterile distilled water. The seeds were resuspended in 2 mL 0.1% agarose and spread onto the surface of a 90×20 mm plate containing primary selective medium (1×MS salt, 1×B5 vitamins, 1% sucrose, 0.5 mg/mL MES (pH 5.7), 30 μg/mL kanamycin, 100 μg/mL carbenicilin, 10 μg/mL benomyl, and 0.8% phytagar). After cold treatment at 4° C. for 3 days, seeds were allowed to germinate and grow for one week at 22° C. under continuous illumination. Due to expression of the NTPII transgene, all transformed seeds germinated and grew into green seedlings, while non-transformed seeds either did not germinate or their seedlings quickly became bleached. Healthy seedlings of the transformants were transferred to and grown on another 90×20 mm plate containing secondary selective medium (comprising the same components as the primary selective medium, except phytagar concentration was increased to 15%) for one week to enhance root development. Finally, these seedlings were transplanted into individual 1″ square pots of Metro Mix soil and grown under standard conditions.

Several thousand T1 seeds of pGYV501, pGYV502, pGYV503, pGYV511, pGYV512, and pGYV513 were subject to selection of primary transformants. This process resulted in 12 transgenic plants for pGYV501, 28 for pGYV502, 29 for pGYV503, 5 for pGYV511, 44 for pGYV512, and 14 for pGY413. During transplanting and growth in soil, some of these transformants failed to survive—probably due to severe levels of transgene expression, disruption of essential genes, or physical damage. The details of the selection are summarized in Table 5.

TABLE 5
Summary of Primary Transformant Selection

| pGYV | Tranformant | Survivor |
|---|---|---|
| Construct | Number | Number |
| pGYV501 | 12 | 8 |
| pGYV502 | 28 | 12 |
| pGYV503 | 29 | 23 |
| pGYV511 | 5 | 5 |
| pGYV512 | 44 | 27 |
| pGYV513 | 14 | 7 |

### Example 4

### Examination of Constitutively Produced DP-1B Fusion Proteins in Leaf Tissues of Arabidopsis Primary Transformants

Example 4 describes: (1) the preparation of leaf protein extracts from pGYV501, pGYV502, and pGYV503 primary transformants, obtained from Example 3; (2) the characterization of the translocated DP-1B fusion protein in pGYV501, pGYV502, and pGYV503 primary transformants; and (3) estimated accumulation of DP-1B fusion protein (as a % of the total soluble protein) in pGYV501, pGYV502, and pGYV503 primary transformants.

Preparation of Leaf Protein Extracts from pGYV501, pGYV502, and pGYV503 Primary Transformants

DP-1Ba, DP-1Be, and DP-1Bv fusion proteins were designed to accumulate in the apoplast, ER lumen, and vacuole of plants transformed with pGYV501, pGYV502, and pGYV503, respectively. Successful translocation would be accompanied by accurate removal of the sporamin targeting determinant peptide sequences from these fusion proteins, thus reducing the sizes of these proteins (approximately) to that of the unmodified DP-1B SLP. Because all DP-1B fusion proteins possessed several repeats of the highly conserved sequence CGAGQGGYGGLGSGGAGRG (SEQ ID NO:29) and a C-terminal 6× histidine tag, fusion protein production could be readily monitored by immuno-blot assays, using DP-1B Abs (WO 9429450) and anti-His (C-term)-HRP (Invitrogen, Carlsbad, Calif.).

Leaf protein extracts were prepared by growing T1 transgenic plants in soil until bolting. One healthy leaf (approximately 30 mg of leaf tissue) from each plant was ground with 50 μL protein extract buffer (50 mM Tris-HCl, pH 8.0, 12.5 mM MgCl₂, 0.1 mM EDTA, 2 mM DTT, 5% glycerol) in a 1.5 mL ice-cold eppendorf tube. The mixtures were centrifuged and the supernatants were collected as leaf protein extracts. Protein concentration of these extracts was determined using Bio-Rad Protein Assay Reagent (Bio-Rad, Hercules, Calif.).

Characterization of DP-1B SLP in pGYV501, pGYV502, and pGYV503 Primary Transformants

Protein immuno-blot assays were used to characterize expression of the DP-1B fusion proteins (as described in Gallagher, S., et al., Current Protocols in Molecular Biology, F. M. Ausubel et al. ed, Wiley Interscience. pp 10.8.1-10.8.21 (1997)). First, 10 μL of leaf protein extract was separated by electrophoresis in a pre-cast 10% mini-polyacrylamide gel (Bio-Rad) and then transferred to a 0.2-μm nitrocellulose membrane (Schleicher & Schuell, Keene, N.H.). The buffers, apparatus and protocols were provided by Bio-Rad. The nitrocellulose membrane was blocked in 5% non-fat milk in TTBS (0.1% Tween-20, 20 mM Tris, 500 mM NaCl, pH 7.5), incubated in DP-1B Abs-TTBS (1:1,000) solution for 3 hr, and in anti-rabbit IgG HRP-conjugate (Promega, Madison, Wis.)-TTBS (1:2,000) solution for 1 hr. Protein-antibody interaction on the membrane was detected by a chemiluminescent substrate solution (100 mM Tris-HCl (pH 8.5), 0.2 mM P-coumaric acid, 2.5 mM 3-aminophthalhydrazide, and 0.01% H₂O₂) and visualized by exposure to ECL Hyperfilm (Amersham Pharmacia Biotech, Piscataway, N.J.). Leaf protein extract made from a well characterized pGY401 (99) plant was used as a positive control (“C”) of unmodified 8-mer DP-1B SLP (WO 01/90389).

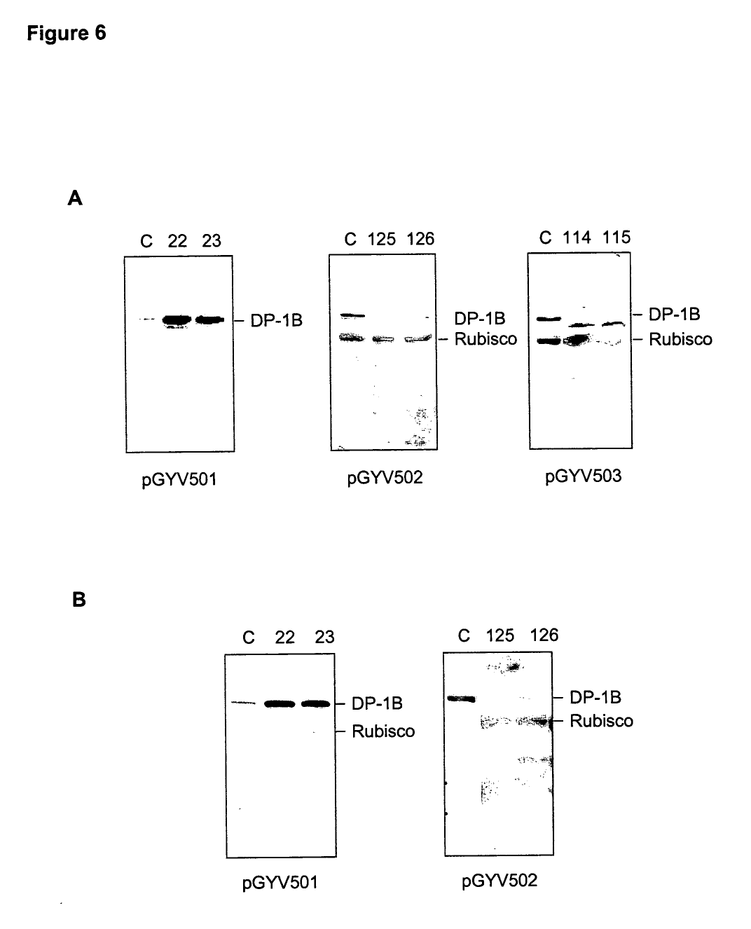

Representative results for pGYV501, pGYV502, and pGYV503 transformants are shown as three separate panels in FIG. 6A (assay results of two individual T1 plants are shown, in comparison to the control “C”). Results demonstrated that the pGYV503 transformants: 1.) did not accumulate intact, processed DP-1Bv fusion protein, and 2.) accumulated DP-1B antibody-reactive protein of the wrong size. For example, both representatives of pGYV503 transformants in FIG. 6A accumulated DP-1 By fusion protein with a molecular size smaller than that of the control “C” DP-1B protein, implying inaccurate removal of the targeting determinant peptides from DP-1Bv fusion protein during vacuole targeting processing. This may lead to further degradation of the entire DP-1Bv fusion protein.

In contrast, results indicated that the Arabidopsis plants transformed with pGYV501 and pGYV502 accumulated DP-1Ba and DP-1 Be fusion proteins with the same size as DP-1B in the “C” sample (FIG. 6A). Although both DP-1Ba and DP-1Be primary translation products were theoretically larger than unmodified DP-1B due to their attached targeting determinant peptides, they appeared identical in size to DP-1B in the positive control “C” (pGY401, 99). Thus, the targeting determinant peptides were trimmed from the DP-1Ba and DP-1Be fusion proteins during protein translocation.

The size reduction of DP-1Ba and DP-1Be fusion proteins could also have been a consequence of peptide removal from the C-terminus rather than the N-terminus, or a result of early termination of translation. To demonstrate that these phenomena were not occurring, a second immuno-blot assay was performed on 20 μL of leaf protein extract from pGYV501 and pGYV502 transformants. Anti-His (C-term)-HRP was used as the primary antibody in a ratio of 1:4,000, and interaction with DP-1B fusion proteins was detected directly by chemiluminescent reagents, without addition of a secondary antibody. Both DP-1Ba and DP-1Be fusion proteins were detected with the anti-His antibody confirming that the C-termini were complete (FIG. 6B). Thus, the sporamin targeting determinant peptides were indeed removed from the N-termini of the DP-1 Ba and DP-1Be fusion proteins during translocation.

Estimation of DP-1B Fusion Protein Accumulation Levels in Leaves of pGYV501, pGYV502, and pGYV503 Primary Transformants

Immuno-blot assay results of FIG. 6 indicated that the accumulation levels of DP-1B fusion proteins appeared to be lower in pGYV502 transformants (ER lumen targeted) and in pGYV503 transformants (vacuole targeted), relative to accumulation in pGYV501 transformants (apoplast targeted). This observation was made since DP-1 B fusion protein signal detection in samples from pGYV502 and pGYV503 transformants required longer exposure to hyperfilm (which resulted in non-specific detection of Rubisco and other smaller proteins in the background).

To determine the exact concentration of DP-1B fusion proteins (i.e., translocated protein accumulation) in the leaves of primary transformants, DP-1B signal from each leaf protein extract (detected by the DP-1B Abs in the first immuno-blot assay, FIG. 6A) was compared with the signal of the positive control pGY401 (99) in the same assay. Because DP-1B concentration in the pGY401 (99) plant (a plant constitutively expressing an unmodified 8-mer DP-1B SLP and accumulating the protein in cytosol) had been quantified previously (92 ng DP-1B in 1 μL of the leaf extract, or 9.2% total soluble protein; WO 01/90389), DP-1B concentration in the leaf protein extracts could be estimated according to the relative strengths of the DP-1B signals. Thus, DP-1B concentrations in the leaf protein extracts were calculated based on DP-1B content and total protein concentration.

The pGY401 (99) transformant was an exceptionally rare event in having the 9.2% accumulation level of un-targeted DP-1B. The pGY401 (99) transformant was found only after screening over 100 transformants as described in WO 01/90389. This is in contrast to the screening of 23 or less transformants with targeting of DP-1B to different locations, wherein some transformants were found to accumulate high levels of DP-1B fusion proteins as described below. Thus for comparison between un-targeted and targeted accumulation levels, the first 16 pGY401 transformants (from WO 01/90389) were compared to the populations of eight to twenty-three pGYV501, pGYV502, and pGYV503 transformants. Concentrations of DP-1B in leaves of the 15 pGY401 transformants were also determined by comparison to the pGY401(99) “C” sample.

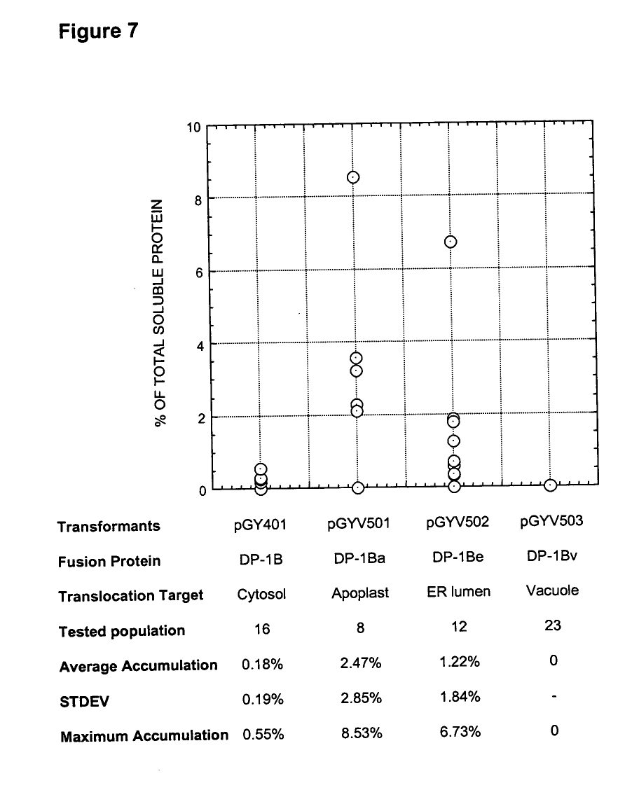

FIG. 7 summarizes the results concerning DP-1B fusion protein accumulation in each primary transformant. A circle represents the DP-1B fusion protein accumulation level in leaf tissue of an individual T1 transgenic plant. As compared to the accumulation of DP-1B at less than 1% TSP in pGY401 transformants where there is no targeting, the results demonstrated that: DP-1B fusion protein accumulation in leaf tissues of transgenic plants was dramatically increased by targeting to the apoplast (average accumulation 2.47% TSP; maximum accumulation 8.5% of TSP);

DP-1B fusion protein accumulation in leaf tissues of transgenic plants was dramatically increased by targeting to the ER lumen (average accumulation 1.22% TSP; maximum accumulation 6.7% of TSP);

Vacuole targeting did not result in accumulation of correct DP-1 B fusion protein (average accumulation 0% TSP);

Thus, both apoplast and ER lumen targeting of the protein greatly increased the chances of identifying transgenic plants with high level accumulation of DP-1B SLP in the leaf tissue. Apoplast targeting was clearly the preferred approach.

### Example 5

### Examination of DP-1B Fusion Proteins in T2 Arabidopsis Seeds

Example 5 describes: (1) the preparation of seed protein extracts from T2 seeds of pGYV511, pGYV512, and pGYV513 transformants; (2) the characterization of DP-1B fusion proteins from these T2 seed extracts; and (3) the estimation of levels of DP-1B fusion protein accumulation in the T2 transgenic seed extracts.

Preparation of Seed Protein Extracts from T2 Seeds of pGYV511, pGYV512, and pGYV513 Transformants

Plants transformed with pGYV511, pGYV512, and pGYV513 synthesized the following fusion proteins in their seeds: DP-1Ba (targeted to the apoplast), DP-1Be (targeted to the ER lumen), and DP-1Bv (targeted to the vacuole), respectively. These seed fusion proteins are subjected to the same processing during translocation as described previously in leaf cells. Specifically, successful translocation of the fusion proteins is accompanied by accurate removal of the sporamin targeting determinant peptide sequences, thereby reducing the size of these proteins to that of the unmodified DP-1B SLP (approximately).

Seed-specific accumulation of the DP-1B fusion proteins was examined by protein immuno-blot assays of the seed protein extracts, prepared from seed of pGYV511, pGYV512, and pGYV513 transformants. To make the seed protein extracts, T1 transgenic plants (from Example 3) were grown in soil until maturation and T2 seeds were individually collected from each of these plants. Approximately 200 seeds from each collection were added to 400 μL protein extract buffer. Seed protein extracts were then prepared using identical methodology to that used for leaf protein extracts and protein concentrations were determined using the Bio-Rad Protein Assay Reagent.

Characterization of DP-1B Fusion Proteins in T2 Seeds of pGYV511, pGYV512, and GYV513 Transformants

Seed protein extracts from T2 transgenic seeds of pGYV511, pGYV512, and pGYV513 transformants, as well as of pGY411 transformants described in WO 01/90389, were subjected to immuno-blot assays (following the methodology of Example 4). DP-1B Abs was applied to detect the highly conserved repetitive sequences of the DP-1B fusion proteins and leaf protein extract made from the well characterized pGY401 (99) plant was used as a positive control (“C”) of unmodified 8-mer DP-1B SLP (WO 01/90389).

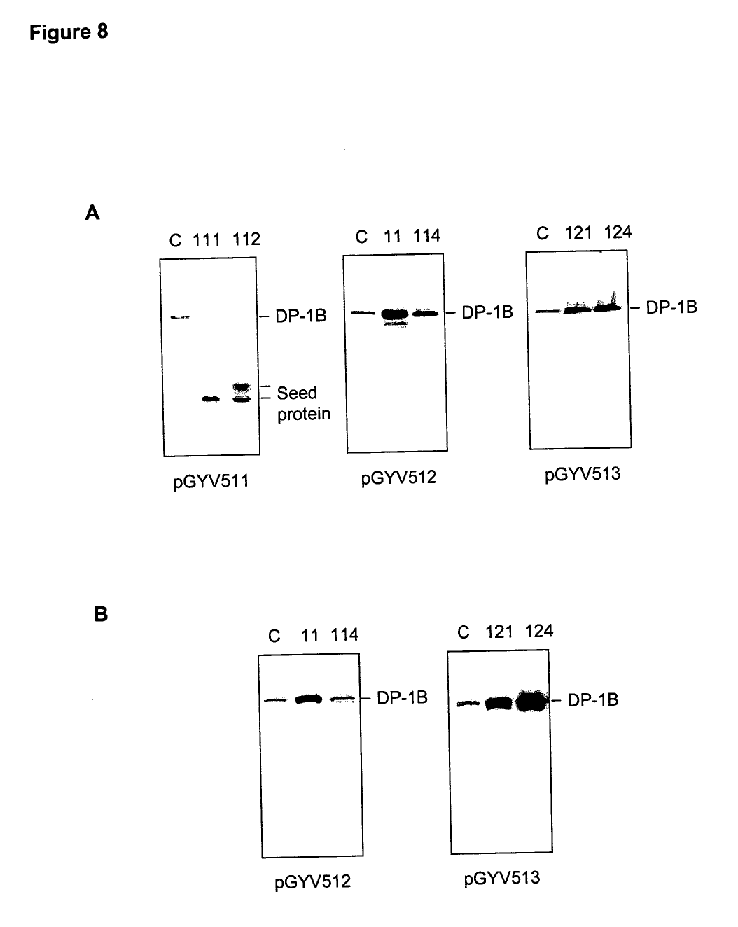

Results indicated that none of the pGYV511 transformants produced DP-1Ba (FIG. 8A). To confirm the lack of DP-1Ba accumulation in seeds, the assays for pGYV511 (sample 111) and pGYV511 (sample 112) were performed over an extended period and developed until nonspecific signals of small seed proteins appeared in the background.

The majority of pGYV512 and pGYV513 transformants accumulated significant amounts of DP-1Be and DP-1Bv fusion proteins in their seeds (see FIG. 8A for representative results). The size of the DP-1 Be and DP-1Bv fusion proteins was similar to the unmodified DP-1B in leaf tissues of the positive control plant pGY401(99) (labeled as “C”), indicating that these fusion proteins had been trimmed to remove the targeting determinant sequences during translocation. Further, a second immuno-blot assay was performed on the seed protein extracts of pGYV512 and pGYV513 transformants using anti-His(C-term)-HRP to directly detect the C-terminal histidine tags (FIG. 8B). Both DP-1Be and DP-1Bv fusion proteins were detected by the anti-His-HRP confirming that the proteins possessed complete C-termini.

Estimation of DP-1B Fusion Protein Accumulation Levels in T2 Transgenic Seeds of pGYV511. pGYV512, and pGYV513 Transformants

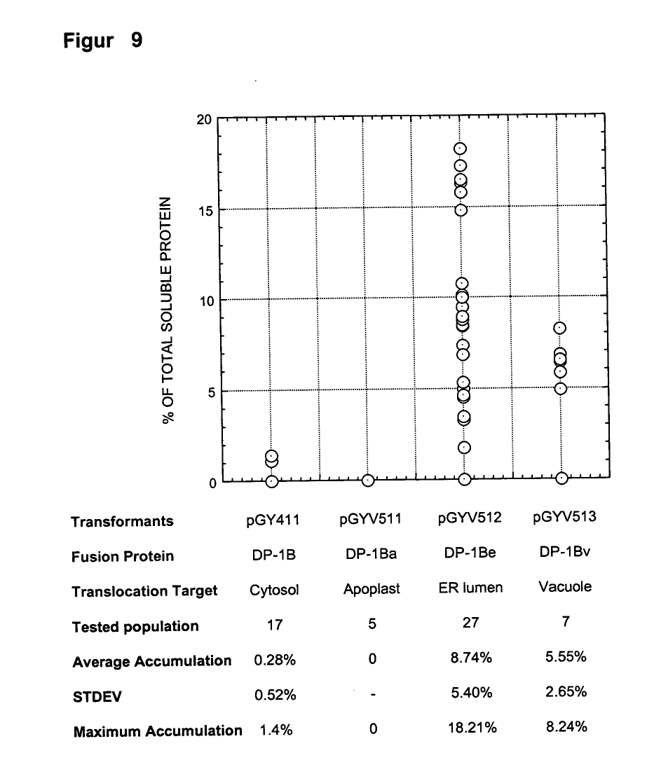

As described in Example 4, production yield of the DP-1B fusion proteins was estimated by comparing the DP-1B signal of each seed protein extract with the signal of the positive control pGY401 (99) (in the first immuno-blot assay; FIG. 8A). Calculations were based on the DP-1B content and total protein concentration of each seed protein extract and are shown in FIG. 9. A circle represents the DP-1B accumulation level in T2 seeds of an individual transgenic plant. Again, data from previous screenings of pGY411 primary transformants (known to synthesize and accumulate an unmodified 8-mer DP-1B SLP in the cytosol of seed cells) was adopted to serve as the control (WO 01/90389). As compared to the accumulation of DP-1B at less than 2% TSP in pGY411 transformants where there is no targeting, the results demonstrate that:

DP-1B fusion protein accumulation in seed tissues of transgenic plants was dramatically increased by targeting to the ER lumen (average accumulation 8.74% TSP; maximum accumulation 18.2% of TSP);

DP-1B fusion protein accumulation in seed tissues of transgenic plants was dramatically increased by targeting to the vacuole (average accumulation 5.55% TSP; maximum accumulation 8.24% of TSP);

Apoplast targeting did not result in accumulation of correct DP-1 B fusion protein (average accumulation 0% TSP);

Thus, an appropriate targeting approach can greatly enhance seed-specific accumulation of the DP-1B fusion proteins. Targeting to the ER lumen is preferred, as it led to the highest accumulation levels.

### Example 6

### Examination of Genetic Hereditability for DP-1B-Derived Transgenes and Their Expression in The Arabidopsis Progeny

Example 6 describes: (1) the preparation of genomic DNA and protein extracts from progenies of the transgenic plants expressing DP-1B fusion proteins constitutively and seed-specifically; (2) the demonstration of DP-1B-derived transgene hereditability (at the DNA level); and (3) the demonstration of DP-1B-derived transgene expression hereditability (at the protein level by examination of protein expression).

Preparation of Genomic DNA and Protein Extracts from Progenies of the Transgenic Plants

To produce the progenies of the selected T1 primary transformants (from Example 3), T2 seeds were collected from each of the T1 plants upon maturation. The T2 seeds were in turn germinated on selective medium. More than fifty T2 seedlings were selected and grown in soil for each parent plant.

Genomic DNA was prepared from the T2 transgenic plants. Briefly, approximately 100 mg of leaves were collected from young seedlings and used to isolate DNA, using DNeasy Plant Mini Kits (Qiagen, Valencia, Calif.). 50 μL of DNA solution was obtained. DNA concentration and purity was estimated by measuring OD₂₆₀ and OD₂₈₀ values in a Beckman DU640 Spectrophotometer (Bechman Instruments, Fullerton, Calif.).

Young leaves were also collected from the T2 plants of pGYV501, pGYV502, and pGYV503 transformants when they began to bolt. And, T3 seeds were collected from the T2 plants of pGYV511, pGYV512, and pGYV513 transformants when the plants became mature. Following the protocols described previously, these T2 leaves and T3 seeds were used to prepare leaf protein extracts and seed protein extracts, respectively.

Demonstration of DP-1B-Derived Transgene Heritability (Confirmation by DNA)

Although accumulation of the DP-1B fusion proteins was previously demonstrated, transgene integration into the plant genome had not been directly confirmed due to limited availability of plant tissue from the primary transformants. Examination of transgenes in the T2 progenies of the transgenic plants would demonstrate not only transgene integration but also transgene heritability.

Due to the highly repetitive nature of the DP-1B coding sequence, the promoter regions and the N-terminal targeting determinant coding regions were among the few unique sequences suitable for primer design for PCR assays. Three PCR primers were synthesized according to these unique sequences: SEQ ID NO:30-32. Primer 35S-F (SEQ ID NO:30) and primer BC-F (SEQ ID NO:31) were forward primers. They were complementary to sequences on the antisense strand of the 35S promoter and beta-conglycinin α′ subunit promoter, respectively. Primer SPM-R (SEQ ID NO:32) was a reverse primer that annealed to the positive strand of the sporamin signal peptide coding sequence. Thus, when paired with primer SPM-R, primer 35S-F or BC-F were used to detect 35S promoter- and BCα′ promoter-containing DP-1B transgenes, respectively.

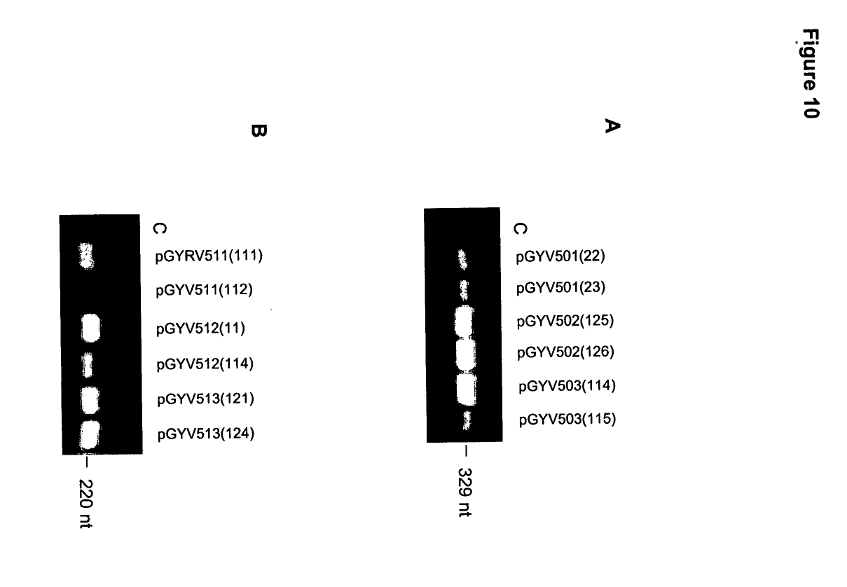

Each PCR reaction included: 1 μL of genomic DNA, 10 pmole of each primer, and 25 μL Ultimate PCR Supermix (Life Technologies). Reactions were pre-heated 5 min at 98° C. and then conducted on a GeneAmp PCR System 960 (Perkin-Elmer, Norwalk, Conn.) for 35 cycles of 30 sec at 94° C., 30 sec at 58° C., and 60 sec at 72° C. PCR products were run on agarose gels and detected with ethidium bromide. Results were compared against a wild type control, labeled “C” in FIG. 10.

PCR assay results indicated that a 329 bp nucleotide transgene fragment was detected from genomic DNA of the pGYV501, pGYV502, and pGYV503 T2 transformants using primers 35S-F and SPM-R (FIG. 10A). A 220 bp transgene fragment was detected from genomic DNA of the pGYV511, pGYV512, and pGYV513 T2 transformants using primers BC-F and SPM-R (FIG. 10B). Thus, integration and heritability of DP-1B-derived transgenes in these plants was confirmed.

Demonstration of DP-1B-Derived Transgene Heritability (Confirmation by Protein Expression)

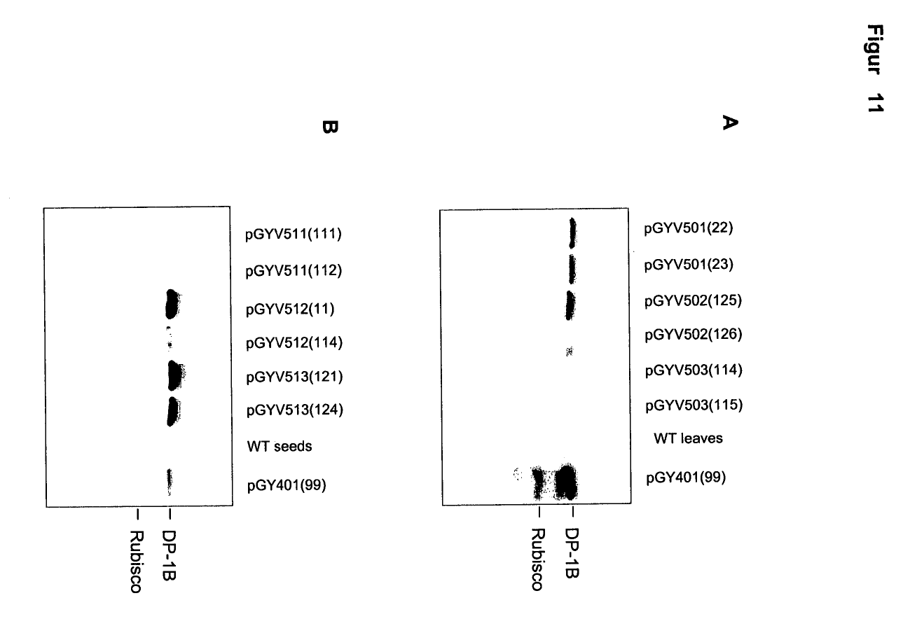

Heritability of transgene expression was examined by protein immuno-blot assays of the leaf or seed protein extracts made from the progenies of the selected transgenic plants (FIG. 11). The positive control was leaf protein extract of pGY401 (99); and wild type leaf protein extract or seed protein extract served as the negative control. Molecular sizes and concentrations of the DP-1B fusion proteins were determined using DP-1B Abs as the primary antibody.

When T2 leaf protein extracts were subjected to the immuno-blot assay, DP-1Ba fusion protein in pGYV501 transformants and DP-1Be fusion protein in pGYV502 transformants were detected, but DP-1Bv fusion protein in pGYV503 transformants was not (FIG. 11A). The accumulated DP-1Ba and DP-1Be fusion proteins possessed molecular sizes equivalent to that of the unmodified DP-1B in the pGY401 (99) control. When T3 seed protein extracts were assayed, DP-1Be fusion protein in pGYV512 transformants and DP-1Bv fusion protein in pGYV513 transformants were detected, but DP-1Ba fusion protein in pGYV511 transformants was not. DP-1Be and DP-1Bv fusion proteins both possessed a molecular size similar to that of the unmodified DP-1B (FIG. 11B). These results are identical to data obtained from the T1 leaf protein extracts and the T2 seed protein extracts. This confirms that expression patterns of the DP-1B fusion proteins were heritable in transgenic Arabidopsis.

DP-1B fusion protein accumulation level in each extract was determined by comparison to the pGY401 (99) control. DP-1B concentrations were calculated based on the signal strength and total protein concentration, as described previously. These results are summarized in Table 6, which directly compares DP-1B accumulation between each parent and progeny. DP-1B accumulation was very similar in different generations for most of the transgenic plants. This was true despite whether the parental accumulation was high, moderate, or low. Exceptions were the transgenic plants of pGYV502 (126) and pGYV512 (114), in which DP-1B accumulation in the progeny was significantly higher than that of the parent. Nonetheless, the assays demonstrated that DP-1B expression and accumulation were heritable in transgenic plants.

TABLE 6
Comparison of Production Yields of DP-1B
SLP between Parents and Progenies

| | | | | Yield in | Yield in |
|---|---|---|---|---|---|
| | | | Trans- | Parent | Progeny |
| Transgenic | Fusion | | location | (% of | (% of |
| plant | Protein | Tissue | Target | TSP) | TSP) |
| pGYV501(22) | DP-1Ba | Leaf | Apoplast | 1.9% | 2.1% |
| pGYV501(23) | DP-1Ba | Leaf | Apoplast | 2.6% | 3.2% |
| pGYV502(125) | DP-1Be | Leaf | ER lumen | 2.3% | 1.8% |
| pGYV502(126) | DP-1Be | Leaf | ER lumen | 1.0% | 6.7% |
| pGYV503(114) | DP-1Bv | Leaf | Vacuole | 0% | 0% |
| PGYV503(115) | DP-1Bv | Leaf | Vacuole | 0% | 0% |
| pGYV511(111) | DP-1Ba | Seed | Apoplast | 0% | 0% |
| pGYV511(112) | DP-1Ba | Seed | Apoplast | 0% | 0% |
| pGYV512(11) | DP-1Be | Seed | ER lumen | 14.4% | 14.7% |
| pGYV512(114) | DP-1Be | Seed | ER lumen | 1.9% | 5.0% |
| pGYV513(121) | DP-1Bv | Seed | Vacuole | 9.8% | 6.9% |
| pGYV513(124) | DP-1Bv | Seed | Vacuole | 7.0% | 8.2% |

What is claimed is:

1. A method for accumulating a translocated protein in a plant tissue comprising:
a) providing a plant having cells comprising a transgene comprising a protein translocation cassette encoding a protein having the general structure: SSP-TP-ER; wherein:
(i) SSP is a sporamin signal peptide;
(ii) TP is a protein to be translocated; and
(iii) ER is an endoplasmic reticulum retention peptide; and
b) growing the plant under conditions whereby the protein translocation cassette is expressed, and the translocated protein is accumulated in the plant tissues.

2. A method according to claim 1 wherein the plant tissues are selected from the group consisting of leaves and seeds.

3. A method according to claim 1 wherein the translocated protein is accumulated in the endoplasmic reticulum of the plant tissues.

4. A method according to claim 1 wherein the translocated protein is accumulated in an amount greater that 1.2% of the total soluble protein.

5. A method according to claim 1 wherein said translocated protein accumulates in an amount greater than 8.7% of the total soluble protein.

6. A method according to claim 1 wherein the endoplasmic reticulum retention peptide has an amino acid sequence selected from the group consisting of SEQ ID NO:33 and SEQ ID NO:34.

7. A method for accumulating a translocated protein in a plant tissue comprising:
a) providing a plant having cells comprising a transgene comprising a protein translocation cassette encoding a protein having the general structure: SSP-TP; wherein:
(i) SSP is a sporamin signal peptide; and
(ii) TP is a protein to be translocated; and
b) growing the plant under conditions whereby the protein translocation cassette is expressed, and the translocated protein is accumulated in the plant tissues.

8. A method according to claim 7 wherein the plant tissues are leaves.

9. A method according to claim 7 wherein the translocated protein is accumulated in the apoplast of the plant tissues.

10. A method according to claim 7 wherein the translocated protein is accumulated in an amount greater that 2.4% of the total soluble protein.

11. A method according to claim 7 wherein the translocated protein is accumulated in an amount greater that 8.5% of the total soluble protein.

12. A method for accumulating a translocated protein in a plant tissue comprising:
a) providing a plant having cells comprising a transgene, the transgene comprising a protein translocation cassette encoding a protein having the general structure: SSP-SProP-TP; wherein:
(i) SSP is a sporamin signal peptide; and
(ii) TP is a protein to be translocated;
(iii) SProP is a sporamin pro-peptide; and
b) growing the plant under conditions whereby the protein translocation cassette is expressed, and the translocated protein is the plant tissues.

13. A method according to claim 12 wherein the plant tissues are seeds.

14. A method according to claim 12 wherein the translocated protein is accumulated in the vacuole of the plant tissues.

15. A method according to claim 12 wherein the translocated protein is accumulated in an amount greater that 5.5% of the total soluble protein.

16. A method according to claim 12 wherein the translocated protein is accumulated in an amount greater that 8.2% of the total soluble protein.

17. A method according to any one of claims 1, 7 or 12, wherein the protein translocation cassette further comprises:
a) suitable regulatory sequences selected from the group consisting of promoters, enhancers, terminators; and
b) optionally, a 6× histidine tag operably linked to C-terminus of the translocated protein.

18. A method according to claim 17, wherein the promoter is selected from the group consisting of:
a) constitutive promoters;
b) tissue-specific promoters;
c) developmental stage-specific promoters;
d) inducible promoters;
e) viral promoters;
f) male germline promoters;
g) female germline promoters;
h) common germline promoters;
i) chemically inducible promoters;
j) plant floral common germline promoters;
k) plant vegetative shoot apical meristem promoters; and
l) plant floral shoot apical meristem promoters.

19. A method according to claim 18, wherein the constitutive promoter is selected from the group consisting of a CaMV 35S promoter, a nopaline synthase promoter, an octopine synthase promoter, a ribulose-1,5-bisphosphate carboxylase promoter, Adh1-based pEmu, Act1, SAM synthase promoter, a Ubi promoter and a chlorophyll a/b binding protein promoter.

20. A method according to claim 18 wherein the tissue specific promoter is isolated from genes encoding the proteins selected from the group consisting of napin, cruciferin, beta-conglycinin, phaseolin, zein, oleosin, acyl carrier protein, stearoyl-ACP desaturase, fatty acid desaturases, glycinin, Bce4, vicilin, and patatin.

21. A method according to any one of claims 1, 7 or 12, wherein the translocated protein is selected from the group consisting of:
a) a transformation marker;
b) a protein conferring a morphological trait;
c) a non-enzyme protein;
d) an enzyme; and
e) a silk-like protein.

22. A method according to claim 21 wherein the silk-like protein is natural or recombinant.

23. A method according claim 21 wherein the silk-like protein is derived from silks produced by Bombyx mori or Nephila clavipes.

24. A method according to claim 22 wherein the silk-like protein comprises the amino acid sequence as set forth in SEQ ID NO:7 and the amino acid sequence as set forth in SEQ ID NO:8.

25. A method according to claim 22 wherein the silk-like protein comprises the amino acid sequences selected from the group consisting of SEQ ID NO:3, SEQ ID NO:4 and SEQ ID NO:5.

26. A method according to claim 23 wherein the silk-like protein is DP-1B.

27. A method according to any one of claims 1, 7 or 12 wherein the plant is selected from the group consisting of food plants, non-food plants, arboreous plants, and aquatic plants.

28. A method according to claim 27, wherein the plant is selected from the group consisting of corn, wheat, barley, oats, sorghum, rice, rye, grasses, banana, soybean, rapeseed, sunflower, cotton, tobacco, alfalfa, Arabidopsis, sugar beet, sugar cane, canola, millet, beans, peas, flax, and forage grasses.

29. A translocation protein cassette encoding a protein having the general structure: SSP-TP-ER; wherein:
(i) SSP is a sporamin signal peptide;
(ii) TP is a protein to be translocated; and
(iii) ER is an endoplasmic reticulum retention peptide selected from the group consisting of SEQ ID NO:33 and SEQ ID NO:34.

30. A protein translocation cassette encoding a protein having the general structure: SSP-SProP-TP; wherein:
(i) SSP is a sporamin signal peptide;
(ii) TP is a protein to be translocated; and
(iii) SProP is a sporamin pro-peptide.


SEQUENCE LISTING (34 sequences)

SEQ ID NO: 1 - protein, 21 aa, Ipomoea batatas
MKAFTLALFLALSLYLLPNPA

SEQ ID NO: 2 - protein, 16 aa, Ipomoea batatas
HSRFNPIRLPTTHEPA

SEQ ID NO: 3 - protein, 101 aa, Nephila calvipes
GQGGYGGLGSQGAGQGGYGGLGSQGAGRGGLGGQGAGAAAAAAAGGAGQGGLGSQGAGQGAGAAAAAAGGAGQGGYGGLGSQGAGRGGQGAGAAAAAAGGA

SEQ ID NO: 4 - protein, 6 aa, Nephila clavipes
SGAGAG

SEQ ID NO: 5 - protein, 6 aa, Nephila clavipes
GAGAGS

SEQ ID NO: 6 - protein, 101 aa, Artificial Sequence; identical to SEQ ID NO: 3
  Peptide sequence for a DP-1B monomer unit

SEQ ID NO: 7 - protein, 34 aa, Artificial Sequence
  Highly repeated consensus motif within a DP-1B monomer unit
GQGGYGGLGSQGAGRGGLGGQGAGAAAAAAAGGA

SEQ ID NO: 8 - protein, 22 aa, Artificial Sequence
  Soft segment within a DP-1B monomer unit
GQGGYGGLGSQGAGRGGLGGQG

SEQ ID NO: 9 - protein, 12 aa, Artificial Sequence
  Hard segment within a DP-1B monomer unit
AGAAAAAAAGGA

SEQ ID NO: 10 - DNA, 34 bp, Artificial Sequence
  Primer SPM+1
atggggaaagccttcacactcgctctcttcttag

SEQ ID NO: 11 - DNA, 45 bp, Artificial Sequence
  Primer SPM+2
ctctttccctctatctcctgcccaatccagcccattccaggttca

SEQ ID NO: 12 - DNA, 35 bp, Artificial Sequence
  Primer SPM+3
atcccatccgcctccccaccacacacgaacccgcc

SEQ ID NO: 13 - DNA, 57 bp, Artificial Sequence
  Primer SPM-1
ggcgggttcgtgtgtggtggggaggcggatgggattgaacctggaatgggctggatt

SEQ ID NO: 14 - DNA, 57 bp, Artificial Sequence
  Primer SPM-2
gggcaggagatagagggaaagagctaagaagagagcgagtgtgaaggctttccccat

SEQ ID NO: 15 - DNA, 114 bp, Artificial Sequence
  Synthetic sequence of sporamin targeting determinant coding region.
atggggaaagccttcacactcgctctcttcttagctctttccctctatctcctgcccaatccagcccattccaggttcaatcccatccgcctccccaccacacacgaacccgcc

SEQ ID NO: 16 - protein, 38 aa, Artificial Sequence
  Synthetic sequence of sporamin targeting determinant coding region.
MGKAFTLALFLALSLYLLPNPAHSRFNPIRLPTTHEPA

SEQ ID NO: 17 - DNA, 20 bp, Artificial Sequence
  Primer SPM-5′
ccaccgccatggggaaagcc

SEQ ID NO: 18 - DNA, 30 bp, Artificial Sequence
  Primer SPM-S
ttgagatctagctggattgggcaggagata

SEQ ID NO: 19 - DNA, 29 bp, Artificial Sequence
  Primer SPM-V
ttgagatctagcgggttcgtgtgtggtgg

SEQ ID NO: 20 - DNA, 83 bp, Artificial Sequence
  PCR amplified sporamin targeting determinant coding sequence SSP suitable for DP-1B fusion protein construction
ccaccgccatggggaaagccttcacactcgctctcttcttagctctttccctctatctcctgcccaatccagctagatctcaa

SEQ ID NO: 21 - protein, 25 aa, Artificial Sequence
  PCR amplified sporamin targeting determinant coding sequence SSP suitable for DP-1B fusion protein construction
MGKAFTLALFLALSLYLLPNPARSQ

SEQ ID NO: 22 - DNA, 131 bp, Artificial Sequence
  PCR amplified sporamin targeting determinant coding sequence SSP-SProP suitable for DP-1B fusion protein construction
ccaccgccatggggaaagccttcacactcgctctcttcttagctctttccctctatctcctgcccaatccagcccattccaggttcaatcccatccgcctccccaccacacacgaacccgctagatctcaa

SEQ ID NO: 23 - protein, 41 aa, Artificial Sequence
  PCR amplified sporamin targeting determinant coding sequence SSP-SProP suitable for DP-1B fusion protein construction
MGKAFTLALFLALSLYLLPNPAHSRFNPIRLPTTHEPARSQ

SEQ ID NO: 24 - DNA, 43 bp, Artificial Sequence
  Primer H6KDEL+
gatcccatcaccatcaccatcacaaggatgagctttaaggtac

SEQ ID NO: 25 - DNA, 35 bp, Artificial Sequence
  Primer H6KDEL-
cttaaagctcatccttgtgatggtgatggtgatgg

SEQ ID NO: 26 - protein, 11 aa, Artificial Sequence
  H6KDEL adapter
SHHHHHHKDEL

SEQ ID NO: 27 - DNA, 43 bp, Artificial Sequence; identical to SEQ ID NO: 24
  Top strand of H6KDEL adapter

SEQ ID NO: 28 - DNA, 36 bp, Artificial Sequence
  Bottom strand of H6KDEL adapter
cttaaagctcatccttgtgatggtgatggtgaatgg

SEQ ID NO: 29 - protein, 19 aa, Artificial Sequence
  Highly conserved sequence within all DP-1B fusion proteins
CGAGQGGYGGLGSGGAGRG

SEQ ID NO: 30 - DNA, 20 bp, Artificial Sequence
  Forward Primer 35S-F
gcctctgccgacagtggtcc

SEQ ID NO: 31 - DNA, 20 bp, Artificial Sequence
  Forward Primer BC-F
cccgtcaaactgcatgccac

SEQ ID NO: 32 - DNA, 22 bp, Artificial Sequence
  Reverse Primer SPM-R
agctggattgggcaggagatag

SEQ ID NO: 33 - protein, 4 aa, Artificial Sequence
  Consensus sequence universally recognized as signals for protein retention in the endoplasmic reticulum (ER)
KDEL

SEQ ID NO: 34 - protein, 4 aa, Artificial Sequence
  Consensus sequence universally recognized as signals for protein retention in the endoplasmic reticulum (ER)
HDEL